(19) United States
(12) Patent Application Publication
(10) Pub. No.: US 2022/0395597 A1
(43) Pub. Date: Dec. 15, 2022

(54) OPTICAL PROPERTIES AND METHODS FOR UV TREATMENT

(71) Applicant: UV Partners, Inc., Grand Haven (US)

(72) Inventor: David W Baarman, Fennville, MI (US)

(21) Appl. No.: 17/771,277

(22) PCT Filed: Mar. 6, 2020
(86) PCT No.: PCT/US20/21437

§ 371 Date: Apr. 22, 2022

Related U.S. Application Data
(60) Provisional application No. 62/924,324, filed on Oct. 22, 2019.

(51) Int. Cl. A61L 2/10; F21V 3/06

(52) U.S. Cl. CPC A61L 2/10; F21V 3/0615; F21V 3/0625

(57) ABSTRACT

A UV light medium can be enhanced to provide specific optical properties that assist in distributing UV light from a UV source to achieve a defined UV light pattern and effective UV disinfection. The properties of the UV light medium can be selected and enhanced by loading the medium with additives, applying a UV blocking pattern, varying the UV light medium material type, thickness, shape, layering, or surface texture. The medium can be any material subject to UV light during a UV treatment or disinfection process. The UV light medium can create a generally uniform UV light pattern that reduces UV hot spots. The UV light medium can diffuse UV light at a target area to enables more robust disinfection with the same or lower UV source intensity.

### BACKGROUND OF THE INVENTION

UV treatment is generally well known. Existing UV treatment solutions range from disinfecting a device in an enclosure to UV-C robotics. Some prior art systems utilize UV transmissive materials to direct UV light toward a disinfection zone. However, known techniques for transmission of UV energy can require high intensities due to the losses experienced during transmission. In addition, these high intensities mean that UV treatment devices are prone to create UV hot spots that can cause damage to the disinfection target or the disinfection device itself.

Many known UV treatment solutions are not designed for use in an open space where the user can be exposed to the UV light. Instead, many known UV disinfection systems are limited to closed systems that are subject to certain limitations that inhibit complete and consistent UV disinfection.

Some known issues of prior UV treatment technologies relate to a lack of understanding of the impact of UV energy on the target being disinfected and the UV treatment device itself. Many UV treatment systems prescribe the “more is better” mantra, which has negative ramifications, especially for materials that are not intended for intense UV exposure. Other issues with prior UV disinfection systems relate to safely and consistently automating the disinfection process.

### SUMMARY OF THE INVENTION

The present invention is generally directed to a UV light medium with enhanced optical properties to assist in distributing UV light from a UV source to achieve a defined UV light pattern. The properties of the UV light medium can be selected and enhanced by loading the medium with additives to some mean particle size distribution of some optical property, applying a UV blocking pattern, varying the UV light medium material type, thickness, shape, layering, or surface texture, or any combination thereof. The UV light medium can be a lens for a UV treatment device, a housing for a device disposed at the target disinfection area, a surface at the target disinfection area, or any other material subject to UV light during a UV treatment or disinfection process. In certain embodiments, the optical properties selected for the UV light medium assist in distributing UV light in a generally uniform UV light pattern. A more uniform UV light pattern can reduce or prevent UV hot spots, which can cause discoloration or other damage from forming on the UV light medium or at the target disinfection area. Certain embodiments provide a UV light medium with diffusal properties that cause the UV light to diffuse or disperse across the surface of the target disinfection area, such as a user interface surface. Diffusing UV light at a surface of the target disinfection area enables more robust disinfection with the same or lower UV source intensity.

In some embodiments, a UV light medium is loaded with UV light altering additives to change the optical properties of the UV light medium. The additives can alter the filtering and reflective properties of the UV light medium to assist in distributing UV energy effectively and safely to a target disinfection area. The additives can provide a light scattering or diffusal effect that assists in disinfection. In one embodiment, the additives are micro-beads with diffusal properties, for example SiO₂ loaded at about 30% by weight into a UV-C transmissive light medium, such as a polymer based UV light medium or TiO₂ loaded at about 30% by weight into a substantially non-UV-C transmissive or opaque UV light medium, such as acrylic plastics or a Formica® counter-top. In alternative embodiments, the additives can have antimicrobial properties, for example the SiO₂ additive can include copper. By loading the UV light medium with SiO₂ with copper, the UV light medium will have increased diffusal properties as well as antimicrobial properties. In another embodiment the purity of glass can be modified using silica engineering to partially enable UVC transmission while reflecting a portion through the surface of the wave guide allowing glass or quartz to have a transmissive portion and a reflective represented as a mean particle distribution of blocking and reflective materials within the glass to reflect UVC energy to and through the surface.

One aspect of the present invention is generally directed to a patterned lens for a UV disinfection device. Some embodiments of the patterned lens prevent UV light hot spots from forming by absorbing or reflecting a portion of the UV light incident with the lens. Accordingly, the pattern can even out the UV light incident with the lens over its surface and prevent the UV light from concentrating toward a certain portion or portions of the lens. Some embodiments, of the patterned lens even out the UV light at an expected target disinfection area. The characteristics of the UV pattern of the lens can be selected depending on a variety of factors, including creating a relatively uniform light distribution through the lens, at a target disinfection area, or both as well as reducing UV hot spots in the lens, target disinfection area, or both. The UV pattern characteristics can include UV blocking pattern shape and size, UV blocking pattern material, UV blocking pattern reflectivity, UV blocking pattern absorption, UV blocking pattern thickness, and UV blocking pattern density. The characteristics of the pattern can be selected to provide a desired UV light output with certain characteristics at a target disinfection area, a desired UV light output with certain characteristics from the lens, or a combination thereof. The characteristics of the pattern can also be selected according to the shape of the UV source, the effects of any reflectors included in the UV disinfection device, a measured UV light output mapping of the UV source, or a simulated UV light output mapping of the UV source in a virtual environment, or a combination thereof. The UV blocking pattern can be applied to the lens in a variety of different ways using a variety of different techniques or combinations of techniques. For example, the UV blocking pattern can be created by joining a material to the lens, such as a thin film, screen, or tape with UV blocking characteristics, or by coating, painting, or etching a pattern with UV blocking characteristics on the lens. Further, material can be removed from a UV opaque film to create a UV blocking pattern joined to a UV transmission lens, such as a UV transmissive film or layer. For example, a pattern of holes can be laser etched into a black plastic UV opaque film to create a desired lens pattern where the remaining material forms a UV blocking pattern that can be paired with a UV transmissive film. It should be noted that by considering UV-C energy patterned distribution in this way the general lighting, human machine interface feedback, and disinfection all follow this design methodology creating the opportunity for multimode lighting and disinfection. In this way, visible light, error and indication status, and disinfection can all be provided through the same optic.

The UV blocking pattern can include zones with different characteristics, for example based on distance to the UV source or the amount and intensity of light incident to a particular zone, in order to achieve a desired UV light output pattern, such as a relatively uniform light distribution through the lens or a light distribution pattern through the lens that generates a relatively uniform light distribution at an expected target disinfection area at a particular distance and orientation from the target. A zone may be limited by resolution of the process used e.g., laser resolution, graphic resolution, molding resolution, etc. In one embodiment, the patterned lens includes a primary zone, for example closest in proximity to the UV light source that receives the highest intensity UV light, along with one or more additional zones, for example located adjacent or outside the primary zone. The characteristics of the UV blocking pattern in the primary and secondary or additional zones can be different and can be selected such that the total amount of UV light passing through each respective zone is below a threshold level that can deform, change, damage or discolor the UV lens or equipment at the target disinfection area during normal use. This also allows a programmable dose of UVC across the surface as prescribed by the zones.

In another aspect, a composite lens with a primary lens and a secondary lens is provided. The primary lens material is less transmissive to UVC than the secondary lens material. The properties of the primary and secondary lens material can be selected by loading the lenses with additives, applying different UV blocking patterns, varying their material type, thickness, shape, layering, or surface texture, or any combination thereof. In one embodiment, the lens material of the primary and secondary lenses may be loaded with the same or different additives in the same or different amounts for loading some or all of the light diffusal, filtering, and reflective properties. The composite lens can reduce or prevent UV hot spots that can deform, change, damage or discolor the UV lens or equipment at the target disinfection area during normal use. The primary lens can provide a first level of UV transmissivity and the secondary lens can provide a second level of UV transmissivity. The primary and secondary lens can be joined, and the position of the secondary lens can be centered or offset with respect to the primary lens. In one embodiment, the secondary lens is an insert that is held in place by the primary lens. In one embodiment, the primary lens includes a pair of fingers that partially or fully surround the secondary lens. The relative position of the primary and secondary lenses can vary depending on the application. In one embodiment, the primary lens is generally cuboid shaped and the secondary lens is generally in the shape of an elliptical cylinder. The composite lens may also include additional lenses.

The composite lens can be incorporated into a disinfection device that can produce generally uniform light distribution at a target disinfection area. The composite lens can be configured with respect to the disinfection device, including the UV source, reflector, and disinfection device housing, to reduce the intensity closer to the UV source and provide an overall energy output pattern to a target distance and dosage with a generally uniform intensity pattern, that is an intensity pattern relatively more uniform than without the composite lens. The generally uniform intensity pattern can be provided even where the distances between the UV source and the target area are not uniform, that is, where certain portions of the target disinfection area are closer or farther away from the composite lens. In one embodiment, the UV source is positioned offset from the target disinfection area and the disinfection device, including the composite lens is positioned at an angle relative to the target disinfection area. The target disinfection area may or may not be a relatively flat surface. For example, the target disinfection area may include a portion of a surface along with any equipment surfaces setting on the surface in the path of the UV light output from the composite lens. The 3D mapping of the target surface may be used to define dosage and intensities for the UVC lighting pattern. The same lens may also be used for general lighting and as an indicator relating to the disinfection status of the surface.

The characteristics of the composite lens, including the amount and type of additives in the primary and secondary lenses, can be selected or adjusted according to the shape and distance of the composite lens from the UV source, the respective distances of the lenses to the target disinfection area, the angle of the composite lens relative to the UV source, the angle of the composite lens relative to the target disinfection area, the shape of the reflector, the distance between the composite lens and the reflector, or a combination thereof. The characteristics can be selected or adjusted according to a measured or simulated UV light output mapping of the UV source. For example, the characteristics can be selected or adjusted based on how the UV light from the UV source travels through the composite lens. The characteristics can also be selected or adjusted based on the pattern of UV light at a target disinfection zone. The system may have multiple varied sources such as UV, general lighting and RGB lighting for indication all using the same or parts of the same lens.

The primary lens may include a supplemental prism that juts inward away from the target disinfection area. The supplemental prism captures additional UV light that otherwise would not be directly incident with the UV source and directs it toward the target disinfection area. The supplemental prism assists in creating a more uniform light distribution at the target disinfection area. For example, the supplemental prism can increase the UV light intensity at areas of the composite lens more distant to the UV source and in turn, more distant locations of the target disinfection zone relative to the disinfection device. This is accomplished by concentrating energy outwardly while reducing energy inward compensating for losses and general optical properties. The supplemental prism may share characteristics with the primary lens or may have a different set of characteristics, including a different type and amount of additives.

Another aspect of the present invention is generally directed to a variable thickness lens for a UV disinfection device. The variable thickness lens provides a generally uniform UV light distribution, which reduces or prevents UV hot spots from forming on the lens or at the target disinfection area. By varying the thickness of the lens material, the amount and intensity of UVC through the lens can be regulated to form a pattern of diffused energy with substantially uniform intensity. The thickness of the lens material affects its transmissive capability; thinner materials generally provide more transmissive capability. In one embodiment, an elongated variable thickness lens can be provided for creating a uniform light distribution for an elongated UV source. The variable thickness lens can have an inner surface closer in proximity to the UV source and an outer surface more distal from the UV source. The curvature of the inner surface and curvature of the outer surface contribute to defining the overall variable thickness of the lens, which provides the generally uniform UV light distribution.

The characteristics of the variable thickness lens can be selected depending on a variety of factors, including creating a relatively uniform light distribution through the lens and reducing UV hot spots in the lens, target disinfection area, or both. The different variable thickness lens characteristics that can be selected can include the curvature of the lens surfaces, thickness of the lens, any additives to the lens materials, and essentially any other characteristics of the lens. Some or all of the surfaces of the lens can be textured to create scattered reflection. Some or all of the surfaces may be coated with a reflector to protect the disinfection device from UV exposure and provide a good dispersion and reflection of the UV light. The surfaces can be both textured and have a reflective coating. The characteristics of the variable thickness lens, including the texture and reflectiveness of the surfaces, can be selected to provide a desired UV output light with certain characteristics at a target area, a desired UV light output with certain characteristics at the lens, or a combination thereof. The characteristics of the variable thickness lens can be selected according to the shape of the UV source, a measured UV light output mapping of the UV source, or a simulated UV light output mapping of the UV source in a virtual environment, or a combination thereof.

The variable thickness lens can include layers with different characteristics, for example based on distance to the UV source or the amount and intensity of light incident to a particular layer, in order to achieve a relatively uniform light distribution through the lens. In one embodiment, the variable thickness lens includes a primary layer, for example closest in proximity to the UV light source or which receives the highest intensity UV light, along with one or more additional layers, for example located adjacent the primary layer or more distal from the UV light source. The characteristics of the layers can be different and can be selected such that the total amount of UV light passing through each respective layer is below a threshold level that can deform, change, damage or discolor the UV lens or equipment at the target disinfection area during normal use. These methods enable configurable optical patterns that can be more homogenous over the surface. This can be desirable for limiting hot spots while reaching desired UV dosages on a surface. The surface may be flat or 3D.

Another aspect of the present invention is generally directed to a housing for a human interface device with UV light distribution. The housing operates in conjunction with an internal or external UV source to distribute UV light to the exposed surfaces of the human interface device for disinfection. The human interface device housing can have optical properties to assist in distributing UV light according to a defined UV light pattern. The properties of the housing can be selected by loading the housing with additives, varying its thickness, applying a UV blocking pattern, varying the type, shape, layering, or texture of materials of the housing, or any combination thereof. The characteristics can be selected or adjusted to provide desired optical properties, such as a desired dosage of UV disinfection. The UV light distribution housing can be integrated into a variety of different human interface devices, such as an elevator interface, a light switch, a keyboard, a mouse, a tube, and a device case.

The various enhancements to the housing can change the light diffusal, filtering, and reflective properties of the housing. The enhancements can be selected and configured to assist in redistributing UV light or energy effectively and safely. The reflective properties can allow portions of the human interface device to have different reflective properties, which allows a varied amount of UV light or energy to move through the surface of the human interface device. These enhancements to the housing can provide a light scattering effect that assists in disinfection.

In another aspect, a UV transmissive skin is provided. The skin can enable transport and distribution of UV light along the skin for disinfection. The skin can be applied to a device, surface, or tubing to enhance distribution of UV energy and disinfection. The UV transmissive skin can have its optical properties selected by loading the skin with UV property altering additives, varying its thickness, applying a UV blocking pattern, varying its material type, layering, shape, or surface texture, or any combination thereof. In one embodiment, the UV transmissive skin surrounds a tube to provide a disinfection skin around the outer surface of the tubing. In another embodiment, the UV transmissive skin is provided at a surface to enhance the UV energy distribution characteristics of the surface. In yet another embodiment, the UV transmissive skin is a multilayer UV disinfection film. The multilayer film includes a transport layer for transmitting UV light along the length of the film and an interface layer for diffusing the UV light from the transport layer to the exposed surface of the interface layer for disinfection. In one embodiment, different layers of the UV transmissive film have different thickness and different loading of additives. The thickness and loading of additives in the interface layer can be such that light received from the transport layer is diffused and reaches the external surface of the interface layer with a sufficient dosage to disinfect the exposed interface surface. The thickness and loading of additives in the transport layer can be such that given a defined UV light input, sufficient light is provided to the interface layer along the entire length of the UV film such that after diffusal by the interface layer, there is sufficient UV light to disinfect the exposed surface, even at the distance farthest from the UV light input. In another embodiment, the UV transmissive skin includes UV transmissive fibers woven into textiles.

In another aspect, a UV-C lens for use in connection with a disinfection device is provided. The lens can seal the opening through which UV-C light is emitted. The lens can have a thin flexible film form factor, for example a fluoropolymer film with UV transmissivity. The lens may be joined with a UV blocking pattern, such as a UV opaque layer. The lens can seal the components within the housing of the disinfection device protecting the components from the environment and the environment from the components, such as dust and water particles. The lens can cooperate with other features of the disinfection device to shape the UV-C illumination pattern output by the disinfection device. For example, the UV-C illumination pattern output by the lens can be cast toward a target disinfection area with various disinfection device structures shaping the UV illumination pattern ultimately cast on the target disinfection area. The shaping can affect characteristics such as the intensity pattern and the overall shape of the UV illumination pattern.

### BRIEF DESCRIPTION OF THE DRAWINGS

FIG. 1 illustrates a UV disinfection device without a UV lens.

FIG. 2 illustrates one embodiment of a lens with a UV blocking pattern.

FIG. 3 illustrates the lens of FIG. 2 installed on a UV disinfection device to enable the UV disinfection device to provide a more uniform UV light distribution pattern.

FIG. 4 illustrates a representative plan view of a UV disinfection device with a composite lens projecting a generally uniform UV light pattern toward a target disinfection area.

FIG. 5 illustrates a perspective view of the composite lens of FIG. 4.

FIG. 6 illustrates a perspective view of a variable thickness lens for providing a uniform UV light distribution pattern.

FIG. 7 illustrates a front view of the variable thickness lens of FIG. 6.

FIG. 8 illustrates a side view of the variable thickness lens of FIG. 6.

FIG. 9 illustrates a representative perspective plan view of a plurality of different target disinfection areas.

FIG. 10 illustrates a graph of distance, intensity, blocking pattern, and dosage.

FIG. 11 illustrates one embodiment of a UV distribution skin in the form of a multilayer UV disinfection film.

FIG. 12 illustrates one embodiment of a UV distribution skin in the form of a UV disinfection film surrounding a tube.

FIG. 13 illustrates one embodiment of an elevator plate enhanced with UV distribution materials.

FIG. 14 illustrates one embodiment of a light switch plate enhanced with UV distribution materials.

FIG. 15 illustrates one embodiment of a keyboard key enhanced with UV distribution materials.

FIG. 16 illustrates one embodiment of a mouse enhanced with UV distribution materials.

FIG. 17 illustrates a perspective view of the variable thickness lens of FIG. 6 installed in a disinfection device.

FIG. 18 illustrates a perspective sectional view of FIG. 17.

FIG. 19 illustrates a side sectional view of FIG. 18.

FIG. 20 illustrates a side sectional view of another embodiment of a lens having enhanced optical properties installed in a UV disinfection device.

FIG. 21 illustrates a perspective view of the lens and disinfection device of FIG. 20.

FIG. 22 illustrates another perspective view of the lens and disinfection device of FIG. 20.

FIG. 23 illustrates a representative side view of an exemplary UV light pattern from a UV source toward a target disinfection area.

FIG. 24 illustrates a representative front view of an exemplary UV light pattern from a UV source toward a target disinfection area.

FIG. 25 illustrates a graph of UV light intensity over the Y axis of the exemplary UV light pattern at the target disinfection area shown in FIG. 23 without a UV lens with enhanced optical properties.

FIG. 26 illustrates a graph of UV light intensity over the X axis of the exemplary UV light pattern at the target disinfection area shown in FIG. 24 without a UV lens having enhanced optical properties.

FIG. 27 illustrates a graph of UV light intensity over the Y axis of the exemplary UV light pattern at the target disinfection area shown in FIG. 23 with a UV lens having enhanced optical properties.

FIG. 28 illustrates a graph of UV light intensity over the X axis of the exemplary UV light pattern at the target disinfection area shown in FIG. 24 without a UV lens having enhanced optical properties.

FIG. 29 illustrates a UV disinfection charging device of one embodiment of the disclosure.

FIG. 30 illustrates a sectional view of a portion of borosilicate glass material with holes in accordance with one embodiment of the disclosure.

FIG. 31 illustrates a top view of a portion of silicon material with holes lens in accordance with another embodiment of the disclosure.

FIG. 32 illustrates a side view of the FIG. 31 silicon material.

FIG. 33A illustrates a representative view of a lens including a UV transmissive layer and a UV opaque layer, the UV opaque layer having a plurality of holes forming a pattern, along with a close-up of a portion of the pattern emphasizing a density of a portion of the holes.

FIG. 33B illustrates a representative view of a lens including a UV transmissive layer and a UV opaque layer, the UV opaque layer having a plurality of holes forming a pattern, along with a close-up of a portion of the pattern emphasizing a different density of a portion of the holes than FIG. 33A.

FIG. 34 illustrates a partial sectional view of FIG. 33A.

FIG. 35 illustrates a perspective view of an embodiment of a disinfection apparatus of the present disclosure.

FIG. 36 illustrates a top view of the disinfection apparatus of FIG. 35.

FIG. 37 illustrates a front view of the disinfection apparatus of FIG. 35.

FIG. 38 illustrates a bottom view of the disinfection apparatus of FIG. 35.

FIG. 39 illustrates a side view of the disinfection apparatus of FIG. 35.

FIG. 40 illustrates a perspective exploded view of the disinfection apparatus of FIG. 35.

FIG. 41 illustrates a representative side view of the disinfection apparatus of FIG. 35 casting a UV illumination pattern toward a target disinfection surface.

FIG. 42 illustrates a side perspective view of the disinfection apparatus of FIG. 35 including an attachment device proximate a keyboard.

FIG. 43 illustrates a top view of the disinfection apparatus of FIG. 35 and UV apparatus.

### DESCRIPTION OF THE CURRENT EMBODIMENT

A. Overview

The present invention relates to improvements associated with distributing UV light, including without limitation various enhancements to an ultraviolet (“UV”) light medium, such as loading additives that change the optical characteristics of the medium, selectively varying the physical characteristics of the UV light medium including the type of material, thickness, shape, layering, or surface texture, application of a UV blocking pattern, or any combination thereof.

UV light can cause a photochemical effect within some polymer and other types of structures, which can cause degradation of the material. As a result, the color of the material can change and the exposed surface can become brittle. Fluoropolymers such as FEP, PFA, and PTFE, and some other polymers are generally resistant or immune to this photochemical effect, at least with respect to UV-C energy in the 100-280 nm range, which is the UV energy often used in UV disinfection/treatment. Lenses, device housings, and user interfaces designed to be subject to UV light can be manufactured out of materials that are resistant or immune to the negative effects of UV light. The properties of these materials can be altered to provide lenses, housings, and user interfaces that are resistant or immune to the negative effects of UV light and also have a set of desired optical properties, such as a desired diffusal level, reflectivity, and UV-C transmissivity. Higher wavelengths from 280 to 400 nm can also be used but may involve longer dosage contact times—the basic principles described herein still apply.

Some embodiments of the present invention relate to various ways to alter optical properties of materials used for disinfecting devices. These materials are designed to improve the device while protecting the user from UV energy. Some of the elements described in this disclosure enable more reliable UV disinfection. Automating or semi-automating disinfection can provide faster and more controlled disinfection—new materials with enhanced optical properties compliment these disinfection systems by providing less destructive and more efficacious solutions. Embodiments of the present invention provide solutions for energy distribution reducing or eliminating areas for bacterial and pathogens to grow while more effectively using the available energy and limiting hot spots across the surface. Some embodiments of the present invention can also limit the destructive forces of UV-C on materials it encounters and also change the UV exposure equation. Some embodiments of the present invention also relate to UV transmissive materials and usage. Lenses and surfaces can provide enhanced UV treatment.

Some embodiments of the present invention leverage the inverse square law, which dictates that the energy toward the center of a UV source has more intense emissions than the energy farther away from the UV source. Some embodiments reduce the UV energy near the center of the UV source being delivered to a surface, for example by selectively blocking, reflecting, or absorbing UV energy. Energy at the center of the lamp can be upwards of 160 μW/cm² or more at the surface while around 6 μW/cm2 at the edges. By reducing the intensity from the 160 μW/cm² range to the 80 μW/cm² range, the UV light exposure time can be increased. That is, the intensity or irradiance of the UV source can be measured in terms of radiometric flux per unit area, sometimes referred to as flux density. The materials can be altered to change the optical properties. For example, using nanoparticles or microparticles the mean size particle distribution can be controlled and the amount of energy that can move through the material can be changed. This can be combined with various sensors and user interface devices such as touch sensors, kiosks, and touch screens. UV light mediums such as quartz, custom glass, plastics, films, and tubing can have their optical properties selected depending on the application and desired UV light distribution. For example, additives can be added and loaded into the UV light medium itself or a coating on the UV light medium, or an absorbing or reflective blocking pattern can be applied or printed to a surface of the UV light medium based on the level and type of optical control desired.

It should be noted that the same design methodologies to provide a lens that outputs a particular UV-C energy pattern distribution can also be utilized to provide specific and tailored general lighting. For example, a visible light medium can have its optical properties selected depending on the application and desired visible light distribution. For that matter, it should be understood that a single light medium can have a set of optical properties selected to provide both desired visible light distribution and UV light distribution. This can be attained by a single blocking pattern that blocks light within both the visible light spectrum (˜380 nm-˜740 nm wavelength) and the UV spectrum (˜10 nm-˜400 nm wavelength). In some embodiments, two different blocking patterns having different optical properties may be joined to the lens—the patterns can be isolated or overlapping and the properties of the patterns at the intersection can be specifically tailored to provide a desired composite set of optical properties. In this way, a light medium that outputs a particular UV-C light pattern distribution and visible light pattern distribution can be provided that enables general lighting, human machine interface feedback, and disinfection all through the same optic or lens. In essence, the optic can be tailored to provide multimode lighting and disinfection. For example, work lights, status indicators, and UV disinfection energy can all be provided through the same lens that includes a blocking pattern effective to limit UV and visible light intensity to desired levels, such as a generally uniform intensity across the lens output for preventing or reducing discoloration or a generally uniform intensity at a target area. As another example, the blocking pattern can be configured to provide the uniform intensity with regard to UV light, while providing a different application with respect to visible light, such as outlining or highlighting a particular area or set of areas, or blocking all but certain wavelengths of light at specific areas of the lens in order to allow transmission of certain colors through the particular areas.

The first inventive aspect of this disclosure is a UV-C lens for cleaning/disinfecting and sealing. UV disinfection devices, such as medical devices, can be rated with IPC ratings for dust and water intrusion. A lens embodiment of the present invention can increase reliability and IPC ratings for dust and water intrusion. The lens can include a thin fluoropolymer film that enhances UV transmissivity and lengthens the life of the disinfection device. While all lenses and lamps of a disinfection device have a finite life with an intensity/transmissivity curve that changes over the life of the device, the current embodiment compensates for those changes by selecting the properties of the lens according to both the UV lamp life as well as the material life. That is, by plotting intensity profiles over the life of the disinfection device, the optical properties of the disinfection device can be selected to compensate for the changes that occur over the life of the lamp and the changes that occur over the life of the device due to the materials used in the design. For example, materials such as fluorinated ethylene propylene (“FEP”) or Silicone MS-1002 can be utilized to manufacture the UV-C lens with the appropriate optical properties that also account for the intensity/transmissivity curve that changes over the life of the device. Some embodiments include a UV-C transmissive lens manufactured from silicon or borosilicate glass. UV transmissive film can act as a lens and seal the components within the housing of the disinfection device protecting the components from the environment and the environment from the components.

The continuum of purity from quartz (generally UV transmissive) to different levels of borosilicate glass (generally UV opaque) enable designer glass that provides a desired level of transmission and internal reflection within the glass. Gorilla® Glass, available from Corning Inc., and other high end compositions, for example, can be designed to have a transmission portion while having an internal reflective portion by enabling the mean particle size distribution of the UV reflective particles to be suspended as a product of design. The second inventive aspect of this disclosure is an additive loaded into the UV light medium to alter the optical properties in a desired fashion. The additive can be particles loaded by weight into the UV light medium. The particles can have different characteristics, such as by filtering, purity, reflecting, or absorbing UV light, for example UVC light. The type, purity, mean particle size distribution, size, density, shape, and amount of the particles loaded into the UV light medium can affect the overall optical properties of the UV light medium. When nanoparticles or micro-particles are used in the UV light medium, such as plastic, various optical properties are provided. The particles, when struck with UV energy, can act to redistribute the UV energy effectively and safely. For example, where the UV light medium is a product subject to UV light for disinfection, the particles can assist in diffusing the UV light around the surfaces of the product. Or, where the UV light medium is a UV opaque or partially UV non-transmissive surface, the particles can assist in diffusing the UV light across the surface. The reflective properties of the particles can allow the UV light medium to have different reflective properties allowing varied energy to move through the surface. The particles can also provide a light scattering effect that assists in disinfection. For example, in one embodiment, 30 micrometer SiO₂ micro-beads can be loaded at 30% by weight into a UV transmissive light medium. As another example, a UV light medium can be manufactured from Silastic™ MS-1002 Moldable Silicone, available from Dow Corning, with a 30% loading by weight of 30 um SiO₂ micro-beads (filler=30%, MOS=70%). The additives can also have antimicrobial properties, for example the SiO₂ additive can include copper or another antimicrobial element. By loading the UV light medium with SiO₂ with copper, the UV light medium will have increased diffusal properties as well as antimicrobial properties.

The third inventive aspect of this disclosure involves limiting UV-C hot spots. UV-C hot spots can deform, change, damage, or discolor the UV lens in a disinfection device or equipment at the target disinfection area. By selecting the optical properties of the UV lens or the material subject to UV light, the UV-C hot spots can be reduced or eliminated. In one embodiment, a UV blocking pattern can be printed on the interior of a plastic lens using UV-C reflective or absorbing materials. This blocking pattern helps to apply inverse square expectations over a surface and distance. One result of the use of the UV blocking pattern, for example printed on the lens, is to reflect unneeded energy from the surface back to other points of treatment. The UV blocking pattern can also serve as a way to evenly age the materials as opposed to having hot spots that age faster and are highly noticeable on light-colored products.

The fourth inventive aspect of this disclosure involves distribution of UV energy, for example UV-C energy. The UV light medium can be enhanced to provide desired distribution of UV energy to a target disinfection area in a specified pattern. In one embodiment, the UV light medium can be enhanced to provide a generally uniform light pattern. The UV light medium can be enhanced in a variety of different ways including, without limitation, loading the UV light medium with additives, varying the type of UV light medium material, thickness, shape, layering, or surface texture, application of a UV blocking pattern, or any combination thereof. In one embodiment, to distribute UV-C evenly through layers of materials and over a surface with varied distances from the lamp involves a thoughtful design process utilizing many tools to best deliver uniform energy over that surface. Thickness of material can also be used to regulate UV-C intensity to form a pattern of diffused energy with substantially uniform intensity. Essentially, the UV light medium can be enhanced to act as a UVC diffuser. One UV light medium can act as a direct diffuser, for example a blocking plastic, or multiple UV light mediums can be layered to provide a desired UVC light diffusal effect in total through the layers. A UV opaque layer can be etched or drilled, for example laser etched or laser drilled with a pattern of holes, to create a desired lens pattern. The UV opaque layer can be paired with a UV transmissive layer, for example any of the UV transmissive layers discussed herein, to provide a multilayer UV lens having a desired set of characteristics and UV transmission properties.

The fifth inventive aspect of this disclosure involves materials for UV distribution. Plastic injected PerFluoroAlkoxy (“PFA”) can be used as a UV light medium for UVC transmission, such as a UV light lens, UV light transmissive housing, or UV light transmissive skin (e.g., a skin that transmits UV light across an outer surface, such as a device or counter-top). Other fluopolymers such as fluorinated ethylene propylene (“FEP”) or polytetrafluroethylene (“PTFE”), can also be utilized as a UV light medium. The thickness of the material is one factor in the transmissive characteristics of the material. Generally, the thinner the material, the more UV transmissive. The surface of the material can be textured to increase UV scattered reflection, for example, the inner surface of a lens may be textured to diffuse or scatter the UV light being input into the lens. The inner surfaces may also be coated with a material having UV reflective properties to protect the device from UV exposure and also provide a good dispersion and reflection of the UV light. In some embodiments, one or more surfaces can be both textured and coated with a reflective material or other UV light altering material. For example, in one embodiment, the UV skin for a counter-top can include additives loaded into the skin to alter the optical properties in a desired fashion. The additive can be particles loaded by weight into the UV skin or the underlying substrate. The particles can have different characteristics, such as by filtering, reflecting, or absorbing UV light, for example UVC light. The type, size, density, shape, and amount of the particles loaded into the UV light medium can affect the overall optical properties of the UV skin. When nanoparticles or micro-particles are used in the UV skin, such as plastic, various optical properties are provided. The particles, when struck with UV energy, can act to redistribute the UV energy effectively and safely. For example, where the UV skin is a product subject to UV light for disinfection, the particles can assist in diffusing the UV light around the surfaces of the product or across the surface. Or, where the substrate is a UV opaque or partially UV non-transmissive surface, the particles can assist in diffusing the UV light across the surface. The reflective properties of the particles can allow the UV skin to have different reflective properties allowing varied energy to move through the surface. The particles can also provide a light scattering effect that assists in disinfection. For example, in one embodiment, 30 micrometer SiO₂ micro-beads can be loaded at 30% by weight into a Formica counter-top. As another example, a UV light medium can be manufactured from Silastic™ MS-1002 Moldable Silicone, available from Dow Corning, with a 30% loading by weight of 30 um SiO₂ micro-beads (filler=30%, MOS=70%). The additives can also have antimicrobial properties, for example the SiO₂ additive can include copper or another antimicrobial element. By loading the UV skin with SiO₂ with copper, the UV skin will have increased diffusal properties as well as antimicrobial properties. For example, a Formica or other non or less-UV transmissive material counter-top loaded with these additives can alter the optical properties such that when UV light is shined on the counter-top the UV light diffuses across the counter-top surface instead of being absorbed or reflected off the surface.

The sixth inventive aspect of this disclosure involves using UV-C transmissive fibers in woven textiles for enhanced UVC disinfection. Polyesters and other plastics are used today in textiles for added stability and wear performance. UV transmissive materials, such as PFA, FEP, and PTFE, can be utilized with common textile materials to create enhanced fibers or filaments with these UV transmissive materials, which provide UV distribution in the completed textile product, such as a lab coat or seating. Using a percentage of these fibers within the textile helps UV light to penetrate the fabric and treat any biological activity trapped within the fabric by light piping UV-C light deeper into the fabric. For example, a UV transmissive fiber or filament can be mixed with other materials like cotton to create a fabric with increased UV transmissive characteristics that enhance disinfection of the product when subjected to UVC light. The enhanced fibers can be made in various sizes for flexibility and wear characteristics.

The seventh inventive aspect of this disclosure involves using reflective additives for UV light distribution. Reflective nano- or micro-particles can enhance UV distribution, and specifically UV-C light distribution. A particular device subject to UV energy for disinfection may contain layers and parts where different factors are balanced in order to maintain proper UV light dosage, safety and exposure parameters, and optical appeal. TO or aluminum particles can be used to reflect UV-C and provide a surface like effect for distribution as well as a filtering effect by proportion.

The present invention is described in the context of various exemplary devices, materials and constructions. It should be understood that the various aspects of the present invention are not limited to illustrative examples provided in this disclosure. Instead, the various aspects of the invention can be implemented in a wide variety of alternative embodiments as described in more detail below. Directional terms, such as “vertical,” “horizontal,” “top,” “bottom,” “upper,” “lower,” “inner,” “inwardly,” “outer” and “outwardly.” are used to assist in describing the invention based on the orientation of the embodiments shown in the illustrations. The use of directional terms should not be interpreted to limit the invention to any specific orientation(s).

U.S. Pat. No. 9,242,018 to Cole et al., which is entitled “PORTABLE LIGHT FASTENING ASSEMBLY” and issued on Jan. 26, 2016; U.S. Pat. No. 9,974,873 to Cole et al., which is entitled “UV GERMICIDAL SYSTEM, METHOD, AND DEVICE THEREOF” and issued on May 22, 2018; International application No. PCT/US2019/023842 to Baarman et al., which is entitled “DISINFECTION BEHAVIOR TRACKING AND RANKING” was filed on Jun. 10, 2019; and International application No. PCT/US2019/036298 to Baarman et al., which is entitled “MOBILE DEVICE DISINFECTION” was filed on Jun. 10, 2019 are incorporated herein by reference in their entireties.

B. Lens with UV Blocking Pattern

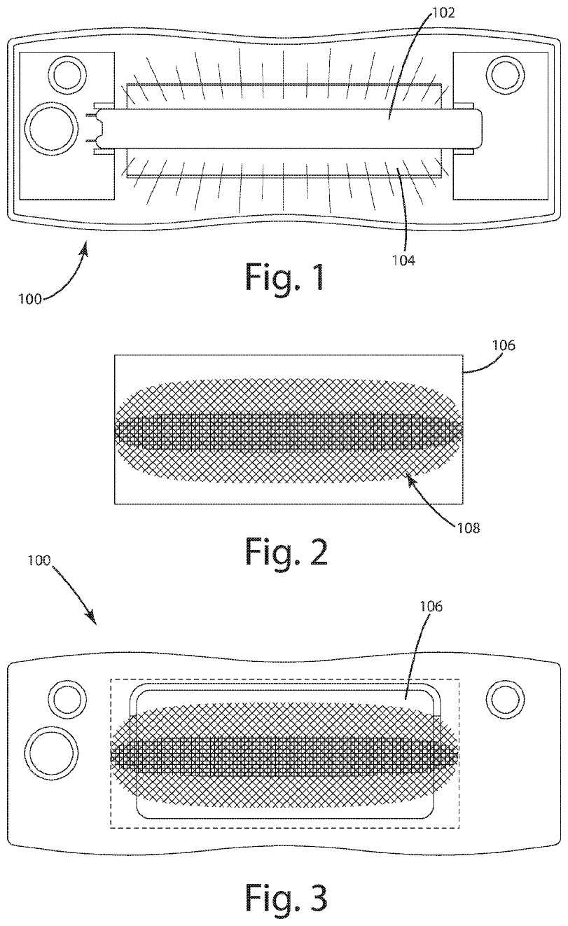

FIGS. 1-3 illustrate an embodiment of a disinfection system for disinfecting surfaces. FIG. 1 illustrates a UV disinfection device 100 having a UV source 102 and reflector 104 without a UV lens installed, FIG. 2 illustrates an exemplary UV lens 106 with a UV blocking pattern 108. FIG. 3 illustrates the exemplary front UV lens 106 installed on the UV disinfection device 100.

The front lens 106 includes a UV blocking pattern 108 that is reflective to UVC for optimizing UVC distribution. In the current embodiment, the UV blocking pattern 108 is a UV transmissive film with a printed pattern that is applied to the internal surface of the lens 106, closest to the UV source when installed. The lens utilizes a printed pattern that is optimized by evaluating the intensity over the surface and limiting the energy in the most intense areas to better balance the energy across the surface. In the current embodiment, the blocking pattern 108 is printed with TiO₂ inks that enable the blocking of energy and reflection of UVC energy back toward the reflector 104.

FIG. 3 shows the lamp and lens system together. By controlling the distribution of energy from the UVC source to precise specifications the exposure results at the target disinfection area are controllable and premature aging of materials caused by the UVC energy can be slowed or eliminated. Further, the blocking pattern 108 can aid in the even distribution of energy. One benefit of evening out the distribution of UVC energy is that the materials age more evenly, making it more difficult to see the effects of aging. Another benefit is that the light distribution of the energy from the lens to the target area is more uniform.

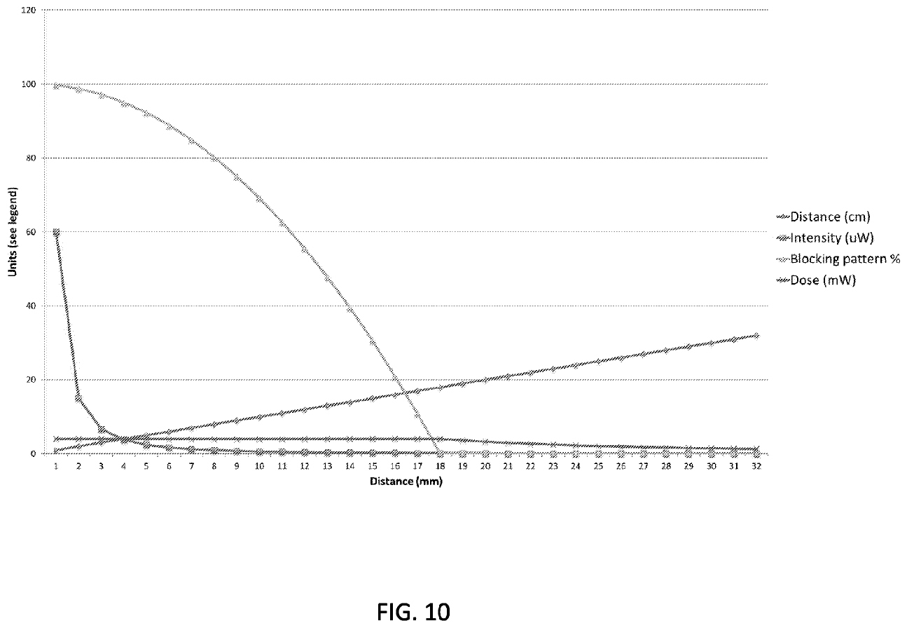

FIG. 10 shows the inverse square law energy drop off of intensity evaluated against filtering pattern percentages to enable even energy distribution for a given distance. That is, FIG. 10 shows the inverse square law as it drops off and how that relates to intensity over distance. The graph also shows an exemplary blocking pattern as it relates to a percentage of energy calculated for a desired distance and pattern of energy. The graph shows that the dose remains relatively flat over a range—that is the lens with the blocking pattern is effectively producing a more uniform UV energy distribution.

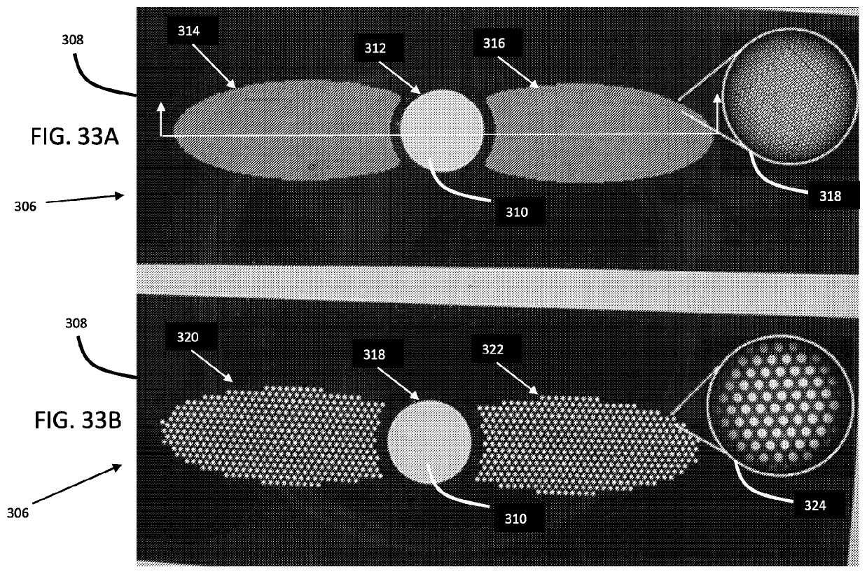

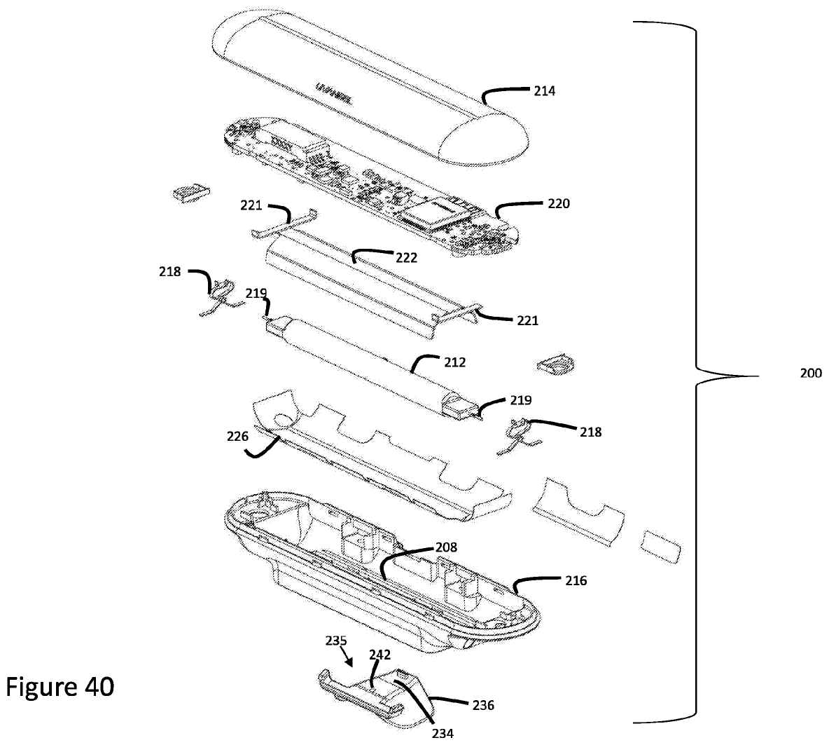

FIGS. 33A-B and 34 illustrate two additional embodiments of an exemplary UV lens 306. Each lens 306 includes a UV blocking pattern that occludes UV-C light (e.g., by reflecting or absorbing UV light) to optimize UV-C light distribution. The UV lens 306 in both the FIG. 33A and FIG. 34B embodiments include two layers, a UV transmissive layer 310 and a UV opaque layer 308. Instead of applying a printed pattern to a UV transmissive layer as discussed in connection with FIGS. 1-3, FIGS. 33 and 33A-B utilize a UV transmissive pattern (e.g., 312, 314, 316 in FIG. 33A and 318, 320, 322 in FIG. 33B) that is selectively removed (e.g., by laser etching) from the UV opaque layer 308. The resulting UV blocking pattern can be optimized by evaluating the intensity over the target disinfection surface and altering the pattern as desired (e.g., to limit UV energy in more intense areas to balance the energy across the target disinfection surface). What remains of the UV opaque layer 308 effectively forms a blocking pattern that blocks UV-C energy or reflects UV-C energy. The resulting lens can be utilized in a disinfection apparatus 200, such as lens 226 shown in FIG. 40. The UV lens 226 can be cut to size as shown in FIG. 40 and can include holes and edges shaped for positioning the UV lens in the disinfection device within the bottom section of the housing 216.

The UV blocking pattern created in the UV opaque layer 308 can be defined by the portions removed from the UV opaque layer 308. In particular, FIG. 33A illustrates a large hole 312, a first plurality of holes 314, and a second plurality of holes 316 being laser etched from the UV opaque layer 308. This can perhaps be seen most easily in the representative sectional view of FIG. 33. Similarly, FIG. 33B illustrates a large hole 318, a first plurality of holes 320, and a second plurality of holes 322 being laser etched from the UV opaque layer 308. In either embodiment, the holes can be laser etched before or after the UV opaque layer 308 is applied to UV transmissive layer 310. The density of the second plurality of holes 316 in the FIG. 33A embodiment are shown in a close-up view 318 and the density of the second plurality of holes 322 in the FIG. 33B embodiment are shown in a close-up view 324. As shown, the density and size of the holes are different in the two embodiments. In particular, the density of the holes in the FIG. 33A embodiment is about twice as sense as the density of holes in the FIG. 33B embodiment. The different densities of holes help to define the UV illumination pattern through the different embodiments of the lens 306.

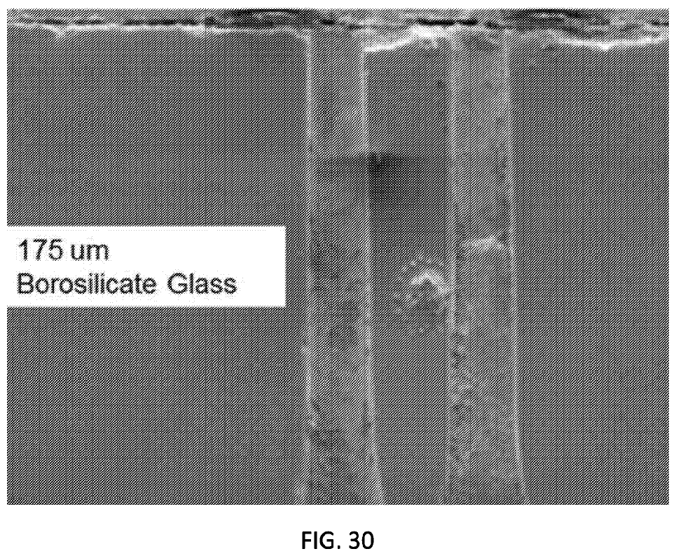

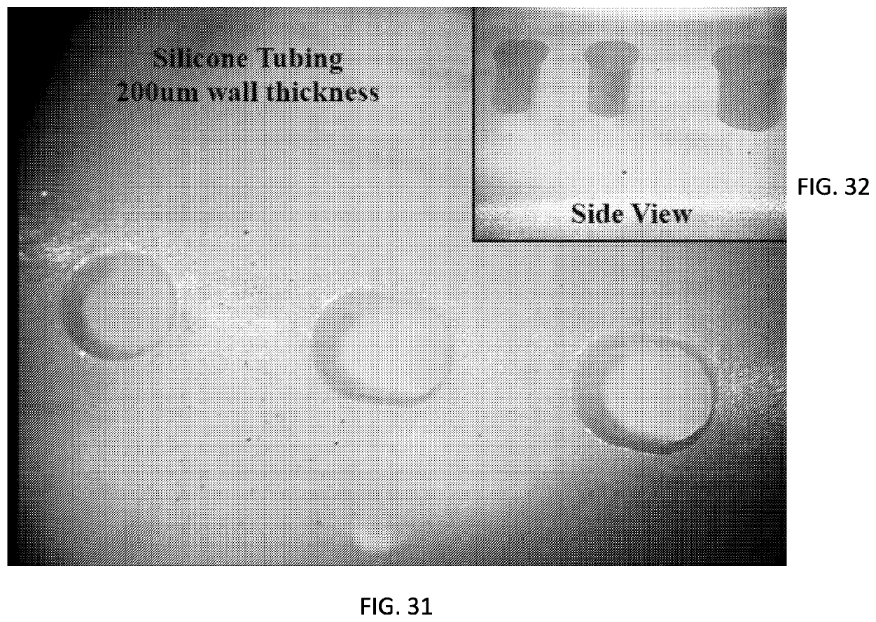

The UV opaque layer can be essentially any UV opaque material. In particular, the UV opaque material may be opaque to light in the entire ultraviolet spectrum (e.g. 10-400 nm). In some embodiments the UV opaque material is opaque to UVA (˜315 nm-˜400 nm) UVB (˜280 nm-˜315 nm) and UVC (˜100 nm-˜280 nm). The UV opacity of the material can depend on the composition of the material and its thickness. The composition and thickness can be adjusted based on a UV transmission curve to ensure that a particular material having a particular thickness is opaque to particular wavelengths. In addition, impurities and additives can also have an effect on opacity and other characteristics of the UV opaque material. Borosilicate glass and silicone tubing are two examples of UV opaque material. FIG. 30 illustrates a sectional side view of borosilicate glass having two holes drilled for UV transmission. The borosilicate has a thickness of about 175 μm. FIG. 31 illustrates a top view of silicone tubing having a 200 μm wall thickness and three holes in the wall. FIG. 32 illustrates a perspective side view of the silicone. In alternative embodiments, a UV partially transmissive layer can be used instead of a UV opaque layer.

C. Composite Lens

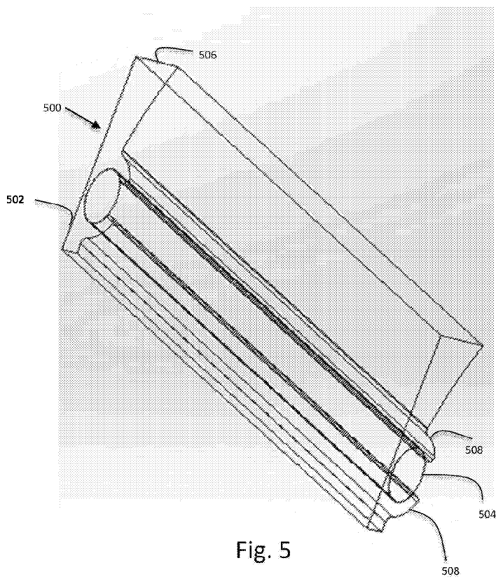

FIG. 5 illustrates a perspective view of an exemplary embodiment of a multipart or composite lens 500. The composite lens 500 includes a primary lens 502 and a secondary lens 504. The properties of the primary and secondary lens material can be selected by loading the lenses with additives, applying different UV blocking patterns, varying their material type, thickness, shape, layering, or surface texture, or any combination thereof. In the depicted embodiment, the secondary lens is made from a material that is less transmissive to UVC than the primary lens materials. Specifically, the light diffusal properties of the secondary lens are greater than the light diffusal properties of the primary lens. Accordingly, UV light passing through the secondary lens tends to diffuse more than UV light passing through the primary lens. These differences, as well as the shaping and positioning of the lenses relative to one another, provide a composite lens that prevents UV hot spots from forming on the lens that can deform, change, damage, or discolor the UV lens.

In the depicted embodiment, the secondary lens 504 is joined to the primary lens in an offset position with respect to the primary lens 502. Perhaps as best seen in FIG. 5, the offset position of the secondary lens, with greater UV diffusion properties, assists in diffusing the UV light and thereby lowering the intensity of the UV light at the target disinfection area. The primary and secondary lens can be joined, and the position of the secondary lens can be centered or offset with respect to the primary lens. In the depicted embodiment, the secondary lens 504 is an insert that is held in place by the primary lens 502. The primary lens includes a pair of fingers 508 that partially or fully surround the secondary lens 504. Further, in the depicted embodiment, the primary lens 502 is generally cuboid shaped while the secondary lens 504 is generally in the shape of an elliptical cylinder.

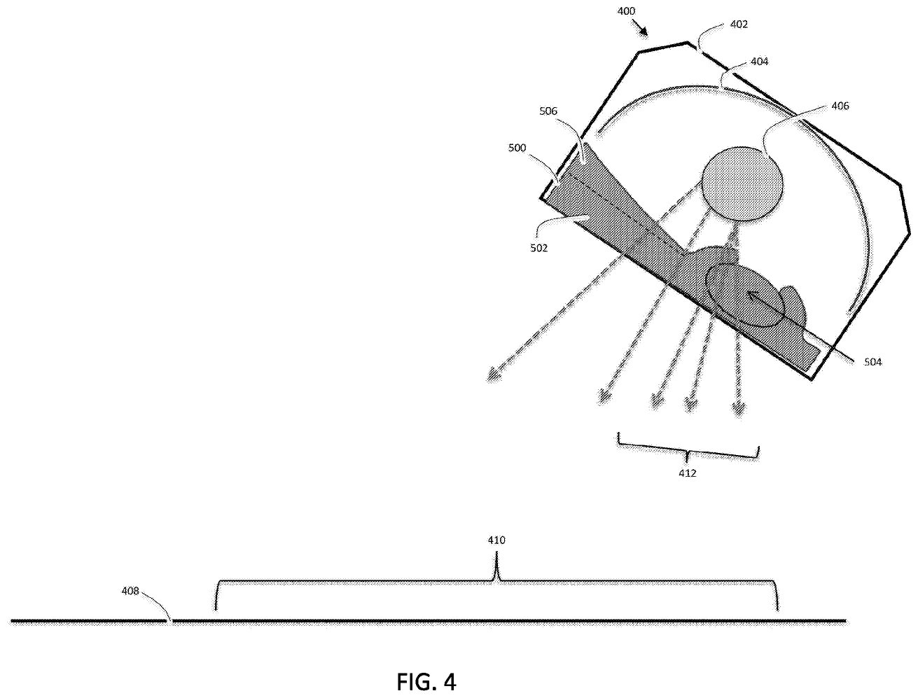

The composite lens 500 can be incorporated into a disinfection device that can produce generally uniform light distribution at a target disinfection area 410. The composite lens 500 can be configured with respect to a disinfection device 400, including the UV source 406, reflector 404, and disinfection device housing 402, to reduce the intensity closer to the UV source 406 and provide an overall energy output pattern to a target distance and dosage with a generally uniform intensity pattern, that is an intensity pattern relatively more uniform than without the composite lens. The generally uniform intensity pattern can be provided even where the distances between the UV source and the target area are not uniform, that is, where certain portions of the target disinfection area 410 are closer or farther away from the composite lens. In the depicted embodiment, the disinfection device 400, including the composite lens 500 is oriented at an angle relative to the target disinfection area. The target disinfection area 410 at surface 408 is relatively flat, however, in alternative embodiments the surface may not be flat. For example, the target disinfection 410 area may include a portion of a surface along with any equipment surfaces setting on the surface in the path of the UV light output from the composite lens. Referring to FIG. 4, the composite lens 500 is shown installed in a UV disinfection device 400. The disinfection device 400 includes a housing 402, a reflector 404, a UV source 406, and as already mentioned, composite lens 500. In the current embodiment, the UV disinfection device is angled relative to surface 408 at about 45 degrees relative to surface 408. The UV disinfection device 400 shines UV light through the composite UV lens 500 toward a target disinfection area 410 located on the surface 408. Due to the positioning and orientation of the UV disinfection device relative to the target disinfection area 410 on the disinfection surface, certain portions of the target disinfection area 410 are substantially closer or farther away from the UV source than others. It is not uncommon for a field disinfection system to have such a configuration due to both logistical issues limiting placement of the UV disinfection device and a desire for the UV light pattern to be shined in a generally downward angle to prevent unintended exposure to UV light. Because the UV light output from the lens is more uniform, it prevents UV hot spots from forming at the target disinfection area. In addition, the positioning and orientation of the UV disinfection device, including the positions and orientation of the primary and secondary lenses 502, 504 relative to the target disinfection area, contribute to providing a uniform UV light pattern output from the UV disinfection device at the target disinfection area 410. Specifically, because the secondary lens 504 causes a more substantial diffusal of the UV light, the intensity through that section produces a heavily diffused lighting area 412, which results in the overall UV light pattern at the UV disinfection area being more even and uniform than if the composite lens were not in place. Put another way, the portion of the target disinfection area 410 closer to the UV source would ordinarily receive a higher intensity UV dosage, however, the properties of the composite lens 500 change the UV light pattern such that the UV light pattern at the target disinfection area is more uninform than it would be otherwise. The UV lens 500 provides a UV light pattern with a highlight diffused lighting area closest to the UV disinfection device along with lesser diffused lighting areas surrounding that more heavily diffused lighting area.

The characteristics of the composite lens 500, including the amount and type of additives in the primary and secondary lenses, can be selected or adjusted according to the shape and distance of the composite lens from the UV source, the respective distances of the lenses to the target disinfection area, the angle of the composite lens relative to the UV source, the angle of the composite lens relative to the target disinfection area, the shape of the reflector, the distance between the composite lens and the reflector, or a combination thereof. The characteristics can be selected or adjusted according to a measured or simulated UV light output mapping of the UV source. For example, the characteristics can be selected or adjusted based on how the UV light from the UV source travels through the composite lens 500. The characteristics can also be selected or adjusted based on the pattern of UV light at a target disinfection zone

The primary lens 500 of the depicted embodiment include a supplemental prism 506 that juts inward away from the target disinfection area. The supplemental prism 506 captures additional UV light that otherwise would not be directly incident with the UV lens 500 and directs it toward the target disinfection area. The supplemental prism assists in creating a more uniform light distribution at the target disinfection area. For example, the supplemental prism can increase the UV light intensity at areas of the composite lens more distant to the UV source and in turn, more distant locations of the target disinfection zone relative to the disinfection device. The supplemental prism may share characteristics with the primary lens or may have a different set of characteristics, including a different type and loading of additives.

D. Variable Thickness Lens

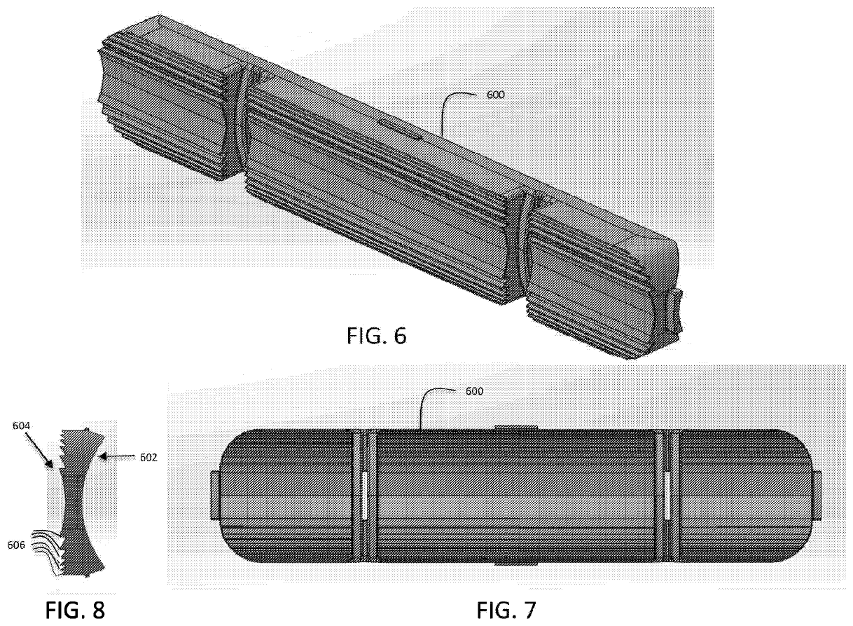

FIGS. 6-8 show an exemplary embodiment of a variable thickness lens 600 that reduces UV intensity by changing the thickness of the lens. FIG. 6 shows a perspective view, FIG. 7 shows a front view, and FIG. 8 shows a side view. In the depicted embodiment, the lens 600 is made from FEP. In alternative embodiments, the lens can be made from a different material, such as Silicone, FPA, PTFE, or other polymers. The thickness of the material reduces the intensity of the UVC. Perhaps as can best be seen in the side view of FIG. 8, the lens has a smooth concave side 602 and a jagged or rough side 604. In the current embodiment, the jagged side 604 is a Fresnel like lens with a series of concentric annular sections. In particular, side 604 has six concave annular sections 606, with decreasing size toward the outer edges of the lens. This configuration assists in providing a more uniform light pattern output from the lens. The optical properties of the lens 600 can be further enhanced to achieve a desired UV light pattern by loading additives into the lens material, such as SIO₂.

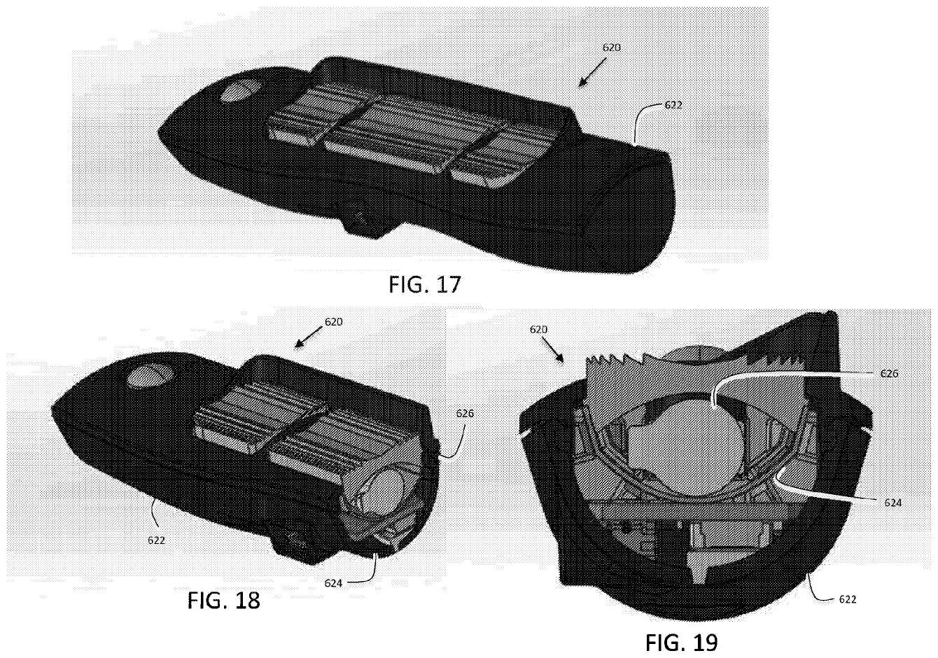

FIGS. 17-19 show a UV disinfection device with the variable thickness lens 600 installed. FIG. 17 depicts a perspective view, FIG. 18 illustrates a sectional perspective view, and FIG. 19 illustrates a side sectional view. The UV disinfection device 620 includes a housing 622, a reflector 624, a UV source 626, and as already mentioned, variable thickness lens 600.

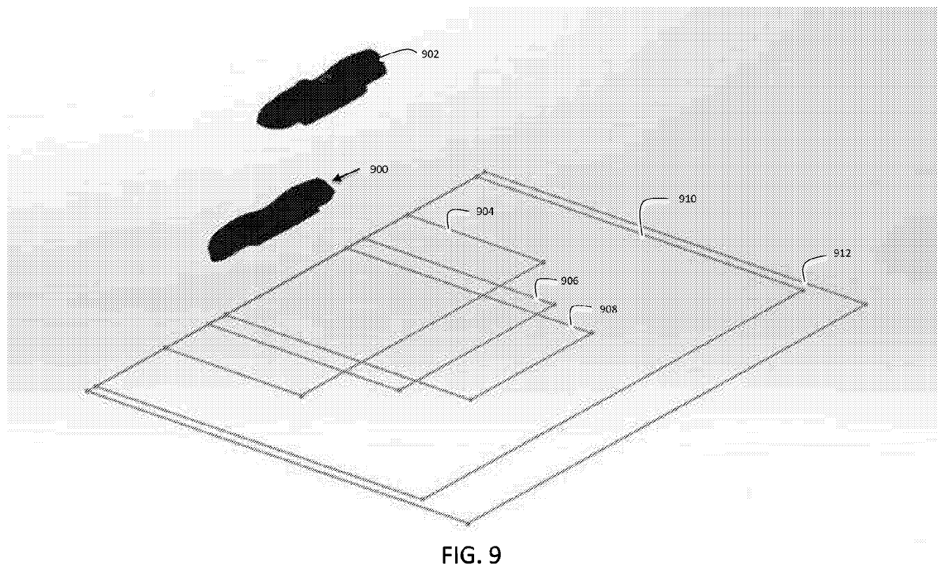

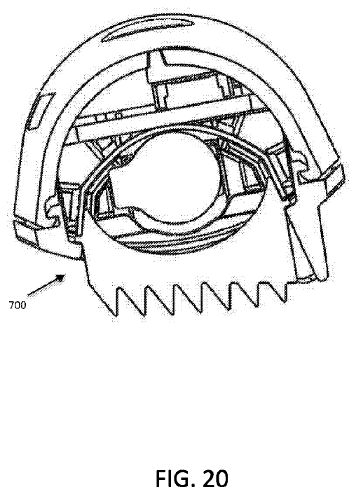

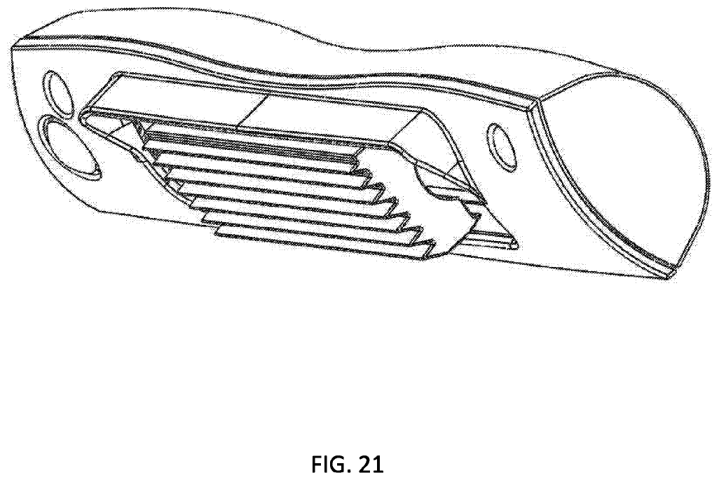

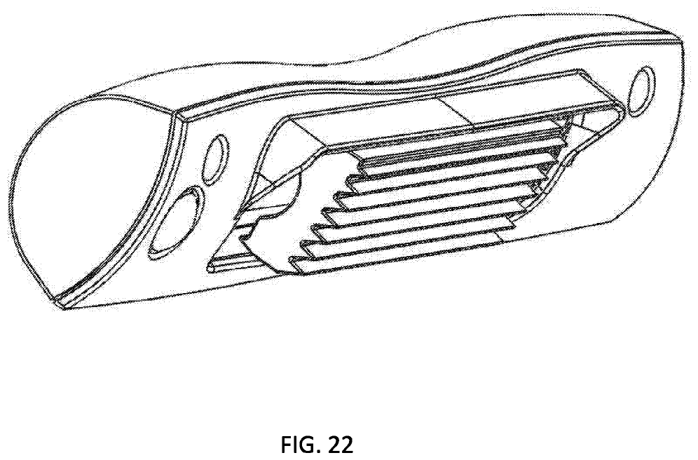

FIGS. 20-22 show another embodiment of a UV disinfection device with a lens 700. FIG. 20 illustrates a side sectional view, FIG. 21 illustrates a perspective view, and FIG. 22 illustrates another perspective view. The variable thickness lens 700 has a different shape than the variable thickness lens of FIGS. 6-8. The outer surface of the lens 700 has a jagged surface that is a series of angled peaks that form a fan-like shape. In the current embodiment, the width of the lens does not reach across the entire UV disinfection device cavity, such that some light unfiltered by the UV lens 700 can escape and contribute to the UV light pattern produced by the UV disinfection device. In alternative embodiments, the width of the lens 700 can span the entire UV disinfection device cavity, such that light unfiltered by the UV lens 700 cannot escape and contribute to the UV light pattern produced by the UV disinfection device. Further, perhaps as best shown in FIG. 20, the orientation and location of the lens with respect to the UV source 706 is such that the certain portions of the UV lens 700 are closer to the UV source area than other portions. These differences, together with the other features of the lens 700, can provide a more uniform intensity pattern while the UV disinfection device is oriented and offset from the target disinfection area, similar to as shown in FIGS. 4 and 9.

One of the features that contributes to the diffusal characteristics of lens 700 are the set of teeth on the output face of lens 700. In the current embodiment, there are seven triangular shaped teeth. The shape, width, orientation, and spacing between the teeth can all contribute to the UV light pattern produced by the UV disinfection device with the lens installed. Another feature that contributes to the diffusal characteristics of lens 700 is its thickness and the curvature of the input and output faces of the lens.

Other features, separate from the characteristics of the lens 700 can also contribute to the diffusal characteristics of the UV light pattern output by the UV disinfection device. For example, the physical location and orientation of the lens 700 relative to the other UV disinfection device components, such as the UV source, the UV source characteristics, the characteristics, orientation, and placement of the UV reflector, if one is included in the UV disinfection device. In addition, the orientation and distance to the target disinfection area also factor in to the intensity mapping of the target disinfection area. In some embodiments, the UV disinfection device may be mounted in a fixed position. In alternative embodiments, the UV disinfection device shade housing, including all of its components may be rotatably mounted. The rotational mount may allow the UV disinfection device to selectably move between a fixed number of orientations. The various features of the UV disinfection device, including the UV source, the lens 700 can be selected such that the UV light pattern produced by the UV disinfection device can provide a desired UV light pattern at a target disinfection area between a minimum and maximum intensity at all of the UV disinfection device fixed orientations. In some embodiments, the UV disinfection device may provide a generally homogenous UV light pattern between a first range of intensities at a first orientation and provide a generally homogenous UV light pattern between a second range of intensities at a second a second orientation. The two ranges may overlap. In this way, the UV disinfection device can provide a generally uniform UV light intensity output pattern to a first target disinfection area and a second target disinfection area where the first target disinfection area is larger than the second target disinfection area.

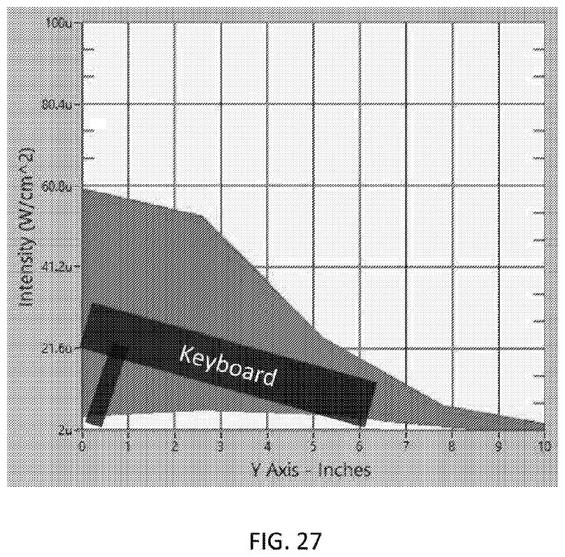

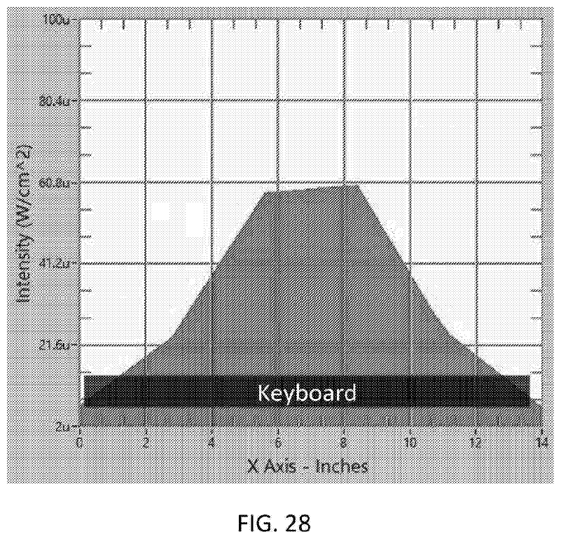

The various features of the lens 700 can be selected such that for a given UV source a threshold maximum intensity is not exceeded ensuring there is no or limited damage/discoloration to the UV disinfection device and any equipment at the target disinfection area. In addition, the various features of the lens 700 can be selected such that for a given UV source a threshold minimum intensity is exceeded to ensure there is sufficient UV dosage to achieve disinfection goals. The various features of the lens 700, in concert with the UV source can cooperate to provide a UV light pattern to a target disinfection area that simultaneously exceeds a UV-C threshold minimum intensity value and also does not exceed a threshold UV-C maximum intensity value. That is, for a target disinfection area, such as the area shown in FIGS. 27-28, the UV light pattern produced by the UV disinfection device can provide a UV light pattern with an intensity in W/cm² over a 14 in (˜5.5 cm) by 10 in (˜3.9 cm) area that is above 2 μW/cm² and below 60 μW/cm². In alternative embodiments, the features of the lens 700 can be selected to provide to provide a UV light pattern between different minimum and maximum thresholds, such as 5 μW/cm² and 70 μW/cm² or 10 μW/cm² and 50 μW/cm².

Referring to FIG. 9, it shows how the optical properties of a lens can be selected to achieve a UV light projection of a pattern of relatively uniform UV energy over a surface. By understanding the distance relationships, the proportional enhancement, or reduction of energy can be used in the lens geometry and materials to impact the uniform projection pattern of the UVC energy. By way of example, the UV disinfection device can be mounted or installed at either position 900 or 902. Position 900 represents a keyboard mounted UV disinfection device or a below monitor mounted UV disinfection device, while position 902 represents an above-monitor or cabinet mounted UV disinfection device. In both circumstances the UV disinfection device is offset from the target disinfection area. The optical properties of the lower mounted UV disinfection device can be selected to provide a UV target disinfection pattern according to any of patterns 904, 906, or 908 by adjusting optical properties of the lens of the UV disinfection device. Similarly, the UV disinfection device 902 can provide a relatively uniform UV disinfection light pattern to the target disinfection area of 910 or 912 by selective adjustment of the optical properties of the lens of the UV disinfection device mounted at position 902.

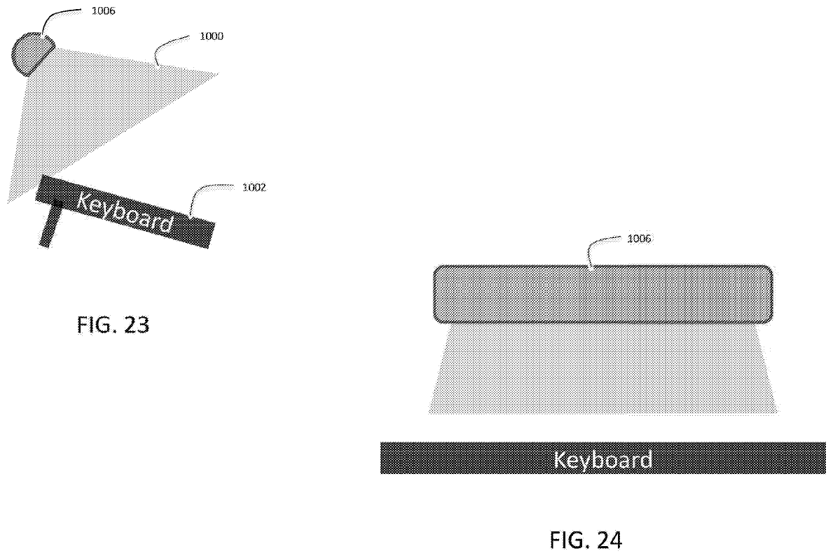

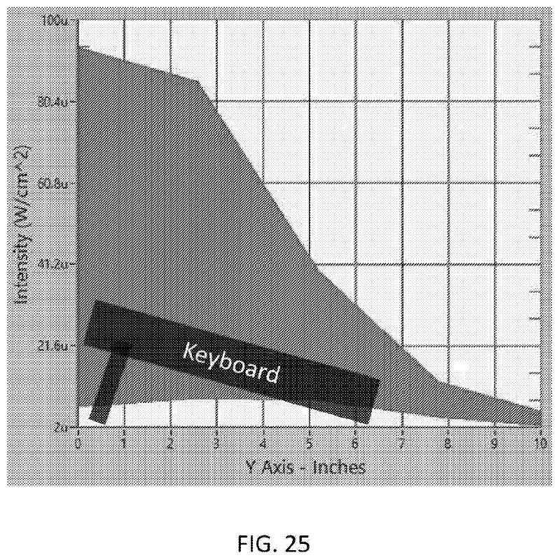

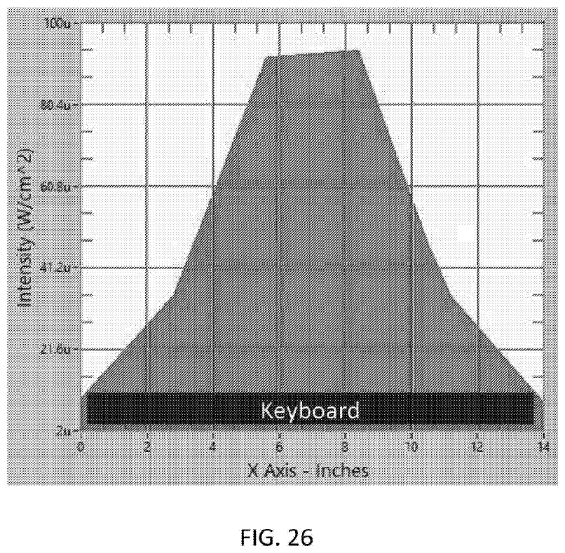

FIG. 9 and the various target disinfection areas can be better understood with reference to FIGS. 23-28, which show a UV light intensity mapping along an exemplary target disinfection area. Specifically, FIGS. 23 and 24 show a representative diagram of a UV light projection pattern 1000 from a UV disinfection device 1006 onto a keyboard 1002 area of a workstation. The UV light projection pattern 1000 is only partially represented to provide the viewer with the general angle and area that is covered by the UV light projection pattern. In practice, the UV light extends to the surface on which the keyboard is disposed, as is evident from the intensity maps of FIGS. 25-28. FIGS. 25-26 show the UV light intensity mapping for an exemplary UV disinfection device without a UV lens. FIG. 25 shows the intensity measurements along the Y axis, while FIG. 26 shows the intensity measurements along the X axis. The keyboard from FIGS. 23-24 is overlaid on the graph to provide a frame of reference. Taken together, the graphs of FIGS. 25-26 illustrate that there is a maximum intensity in this example of about 95 μW/cm² in the center of the keyboard directly under where the UV disinfection device is disposed. The UV light intensity falls off in both the Y and X axis as the UV light reaches further away from the UV disinfection device. FIGS. 27-28 illustrate the effects of the UV lens of FIGS. 20-22 on the intensity mapping. With the lens of FIGS. 20-22 installed, the UV light pattern is more uniform across the target disinfection area. Taken together, the graphs of FIGS. 27-28 illustrate that there is a maximum intensity of about 61 μW/cm² with the UV lens installed, which is considerably lower than the 95 μW/cm² maximum without the UV lens.

E. UV Transmissive Film

A UV transmissive film can be applied to a device or surface to improve UV disinfection of the device or surface. The optical properties of the UV transmissive film can be selected by loading the film with additives, applying a UV blocking pattern, varying the film material type, thickness, shape, layering, or surface texture, or any combination thereof.

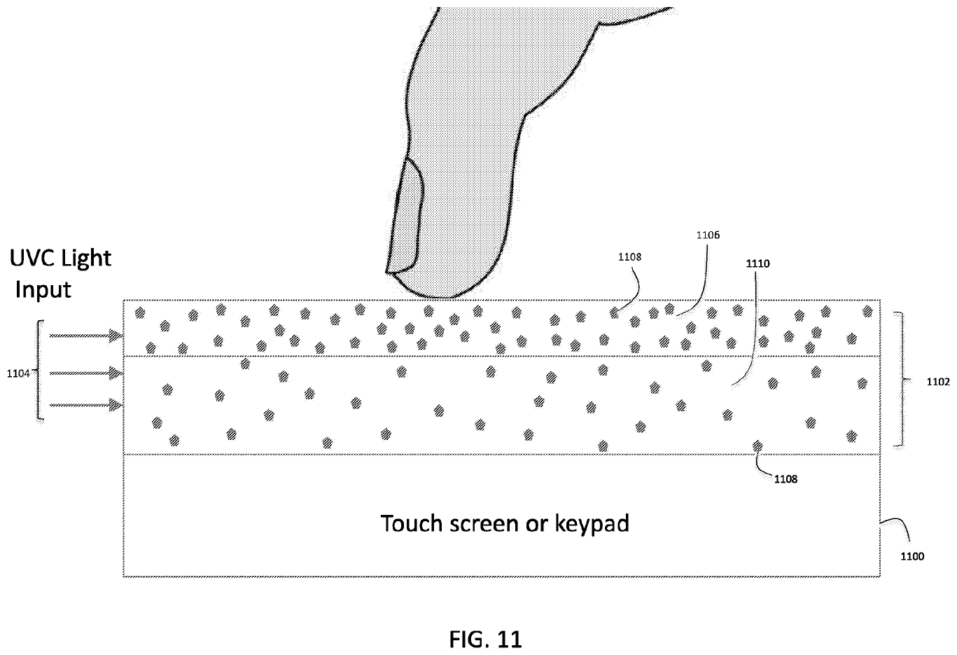

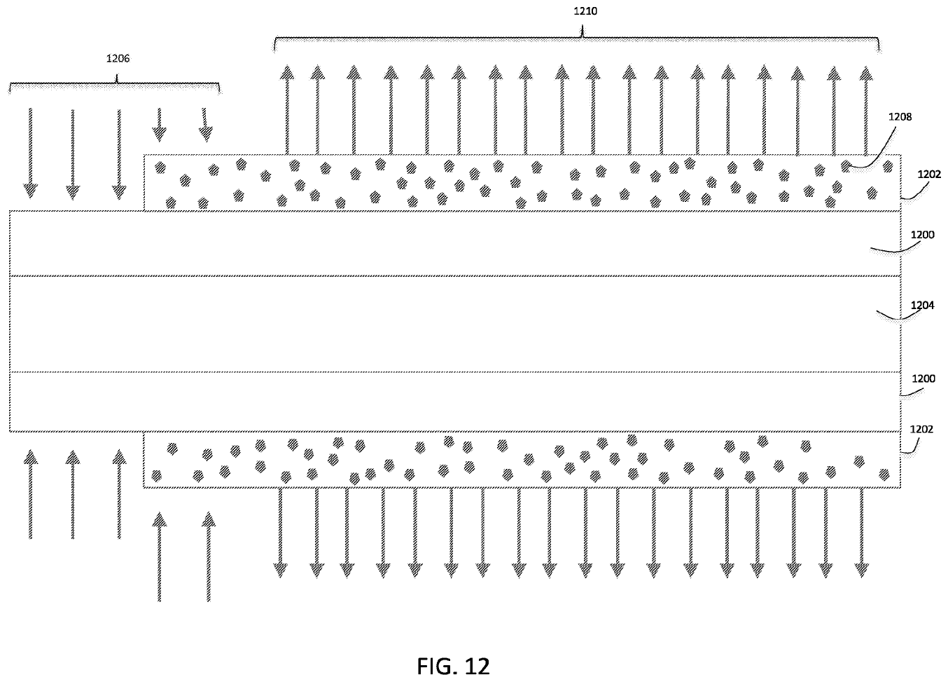

In one embodiment, depicted in FIG. 11, a multilayer UV transmissive film 1102 is applied to the surface of a touch screen or keypad 1100 of a human interface device, such as a tablet, computer, kiosk, or other human interface device. UV input light 1104 may be directed toward the edge of the multilayer UV transmissive film 1102 from a UV source (not shown) either installed on or within the human interface device or external to the human interface device. The UV input light 1104 may be incident with the edge of the entire multilayer UV transmissive film 1102 or with only a portion of the UV transmissive film.

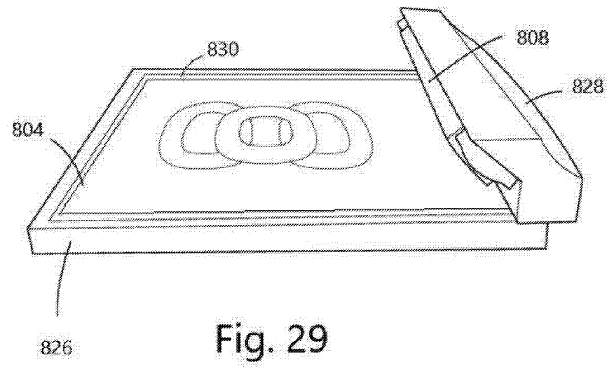

In another embodiment, depicted in FIG. 29, the multilayer UV transmissive film can be applied to a substrate or form the surface 804 of a UV disinfection charging device, such as the UV charging rack or UV disinfection charger described in International application No. PCT/US2019/036298 to Baarman et al., which is entitled “MOBILE DEVICE DISINFECTION” was filed on Jun. 10, 2019, and is incorporated herein by reference in its entirety. The UV disinfection charger depicted in FIG. 29 includes a UV source 808, a UV shade 828 for the UV source, a UV multilayer transmissive surface 804 and a UV disinfection charger housing 826. The details regarding the charging, for example charging electronics for wired or wirelessly charging a device disposed on the UV multilayer transmissive surface 804 are conventional and therefore will not be described in detail. The UV multilayer transmissive surface may include markings or other features to indicate where one or more devices should be positioned or disposed on the surface 804.

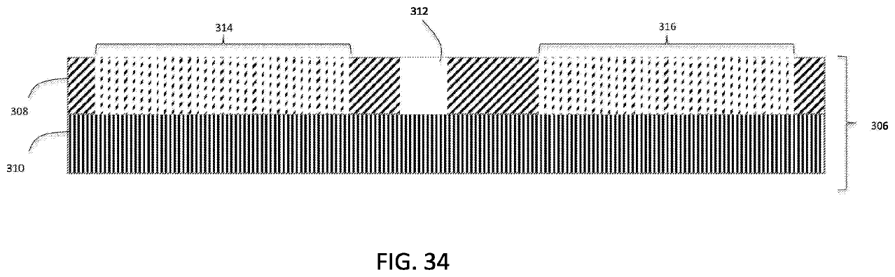

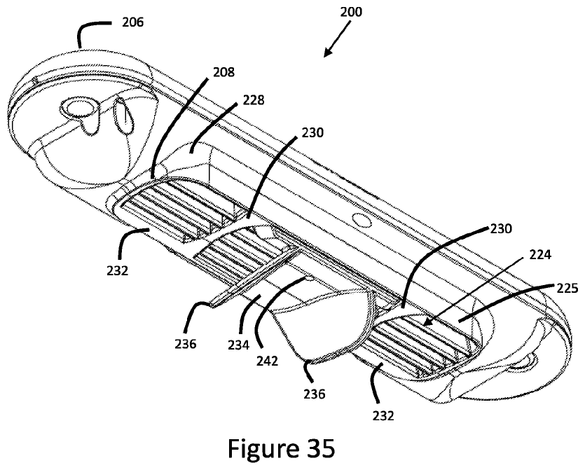

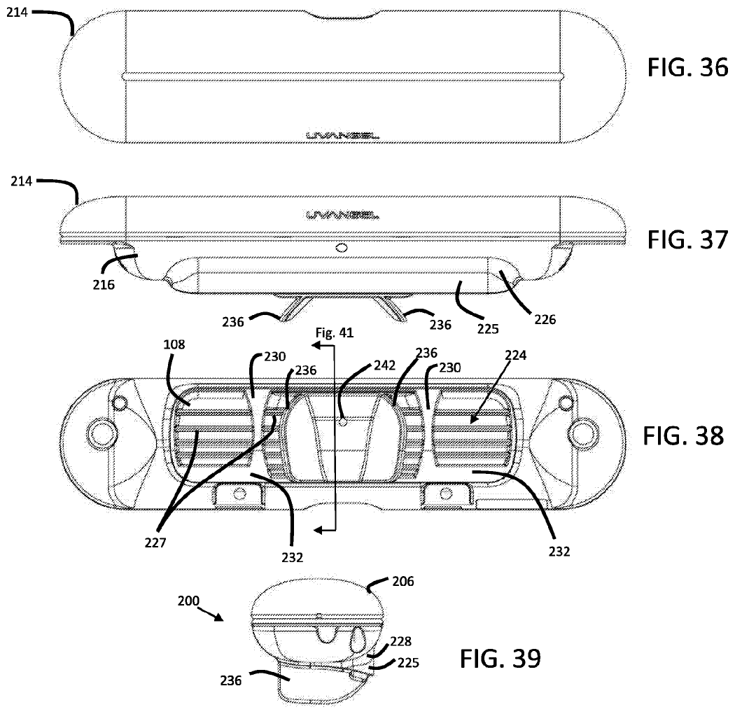

In another embodiment, depicted in FIG. 35-43, a UV transmissive film 226 is disposed between a UV source 212 in a housing 206 of a disinfection apparatus 200. The UV transmissive film 226 can be a multilayer lens. Typically, the disinfection apparatus 200 is configured for at least partially disinfecting a target disinfection area such as a surface. For example, the apparatus 200 can be configured to disinfect a human interface surface of a device or other piece of equipment, which can include a human touch surface. The disinfection apparatus 200 can include a housing 206 that defines an aperture 208. The housing 206 can include a first, top, portion 214 and a second, bottom, portion 216 that are configured to be joined together to form the housing 206.

Perhaps as best shown in the exploded perspective view of FIG. 40, the disinfection apparatus 200 can further include an ultra-violet (UV) light source 212 that can be at least partially enclosed in the housing 206. The UV light source 212 can be retained in place within the housing by two retaining clips 118 that physically fix the UV light 212 in place within the housing and also electrically couple the terminals 219 of the UV light source to a circuit board 220. A reflector 222 can also be disposed within the housing 206.

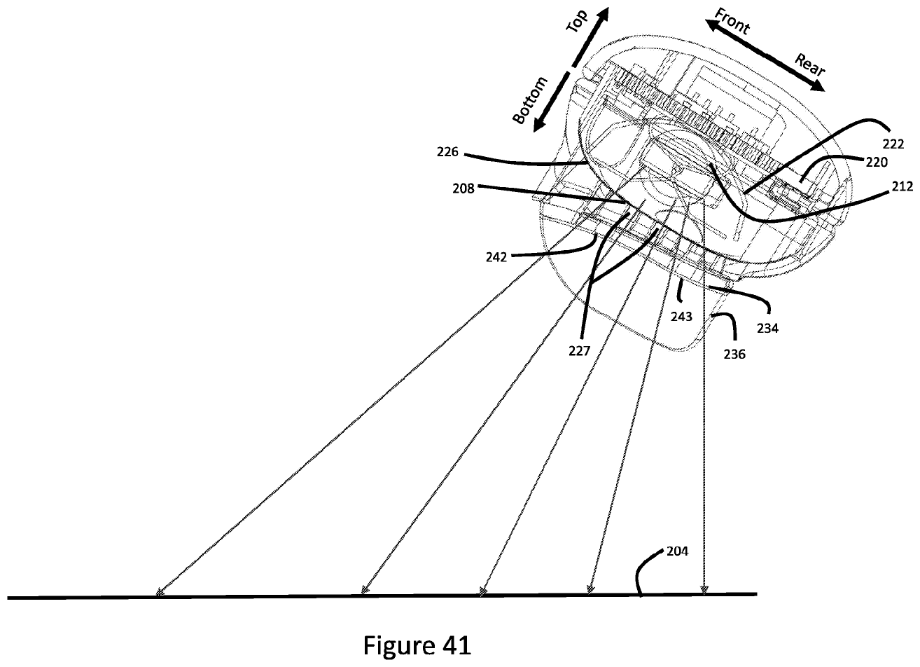

The UV light source 212 can project an illumination pattern toward a target disinfection area 204. The disinfection device 200 can be configured such that the UV light source 212 projects an illumination pattern that substantially corresponds to an expected target disinfection area 204, such as the touch surface of a human interface device, for example keyboard 202 as shown in FIG. 41. The UV illumination pattern can be influenced and defined, at least partially, by the aperture 208. For example, the aperture 208 of the depicted embodiments is generally stadium shaped and the elongated UV light source 212 is generally aligned both longitudinally and laterally with the opening of the aperture 208 such that the illumination pattern is generally restricted to the general shape of the aperture. However, because the disinfection device 208 is typically offset and raised relative to the target disinfection area 204, for example as shown in the embodiments of FIGS. 41-43, the disinfection device casts a UV illumination pattern at an angle relative to the expected target disinfection surface. As such, while the UV illumination pattern through the aperture 208 (without considering the other UV pattern influences) is a generally stadium shape that is elongated at the target disinfection area due to the orientation and position of the disinfection device relative to the surface the pattern is being cast upon. In addition, the intensity distribution of the UV illumination pattern generally forms an elliptical shape where the intensity of the UV illumination pattern maps to the elongated shape of the UV source and fades laterally and longitudinally, falling off quicker toward the longitudinal edges due to the elongated shape of the UV source, which provides an increased intensity along its lateral axis.

The UV illumination pattern can also be influenced and defined, at least partially, by any optics in the path of the UV light projection, such as reflector 222, lens 226, louvers 224, louver frame 228, eyebrow 225, nose 235, or any combination thereof. The optics, either alone or together in various combinations, can perform a variety of different functions including UV pattern control, UV pattern shaping, UV pattern extension, UV pattern redirection, UV pattern exclusion, UV intensity limiting, UV intensity smoothing, UV line of sight limiting, and UV dosage control. These functions can be achieved by forming the various components from UV transmissive, UV transparent, UV reflective, UV opaque materials, or combinations thereof, such as various polymer, metal, composites, or other materials. The optics can perform these various functions in a variety of different ways, for example by obstructing UV light, reflecting UV light, refracting UV light, absorbing UV light, redirecting UV light, occluding UV light, or any combination thereof. The UV illumination pattern received at the opening 208 after passing through the UV lens 226 can be occluded by one or a combination of multiple different optical occlusions positioned within the opening 108 including the louvers 227, louver frame 228, eyebrow 225, occlusion plate 234, and reflective fins 236. That is, the UV illumination pattern output by the disinfection device can be shaped by a UV-C illumination pattern shaping system that extends from the opening 208. The UV-C illumination pattern shaping system can include one or more of louvers 127, louver frame 128, eyebrow 125, occlusion plate 134, and reflective fins 136. Specifically, the UV-C illumination pattern shaping system can receive UV-C light from the opening 108 and shape the UV-C illumination pattern into a shaped UV-C illumination pattern for casting on to an expected target disinfection area or surface. The shaped UV-C illumination pattern can be shaped to have characteristics such that when cast onto the expected target disinfection surface or area the resultant UV-C illumination pattern on the area or surface has a generally uniform intensity. That is, the shaped UV-C illumination pattern characteristics account for the orientation and position of the disinfection device relative to the expected target disinfection area and the disinfection device includes optic features, such as the UV-C illumination pattern shaping system extending from the housing to adapt the UV light to provide the shaped UV-C illumination pattern such that when cast on the expected target disinfection surface at the expected distance and position relative to the disinfection device, the UV illumination pattern is relatively uniform.

The uniformity of the intensity of a UV light pattern cast by a disinfection device can vary depending on a number of different factors. Two such factors are the characteristics of the UV light pattern output from the disinfection device and the distance to the target disinfection area. It is worth noting that the contour of the target disinfection surface can affect the distance and therefore the ultimate intensity at the target disinfection area. Distance is a factor because of the inverse square law, as discussed herein, which essentially states that illumination intensity changes in inverse proportion to the square of the distance from the source. In simple terms, for a given illumination pattern, as the distance from the source doubles, the light intensity falls off by four times. This means that for a plane adjacent to an omnidirectional light source, the light pattern on the plane will tend to have highest intensity where the light source is closest and then quickly fall off in all directions away from that point because as distance between the light source and the plane increases, the intensity of the light will drop.

In practice, the intensity of a UV illumination pattern is more complex. The UV source may not be omnidirectional and the target disinfection area likely is not a plane adjacent to the source. The UV source may include multiple discrete sources, the shape of the source may be elongated, the UV light may interact with a reflector, a lens, an occlusion, directional louvers, or a combination thereof. For example, where the UV lamp is elongated, the UV light pattern tends to have highest intensity in the middle, with the intensity fading quicker in the longitudinal directions than in the latitudinal directions due to the elongated shape of the lamp. Further, the UV source may be offset and cast its pattern at a downward angle toward a target disinfection area. The target disinfection area may itself have a varied contour, such as a keyboard, mouse, or other type of irregular surface. Further, the optical properties of the lens can have a meaningful influence on the path of the UV light. Accordingly, to provide a relatively uniform intensity at a target disinfection area, the UV illumination pattern output from the disinfection device likely will have a non-uniform intensity pattern—and more particularly, a UV illumination pattern with a non-uniform intensity selected such that the UV illumination pattern once it reaches an expected target disinfection area will have a generally uniform intensity given the expected target disinfection area being within a certain distance with respect to the disinfection device and certain orientation with respect to the disinfection device.

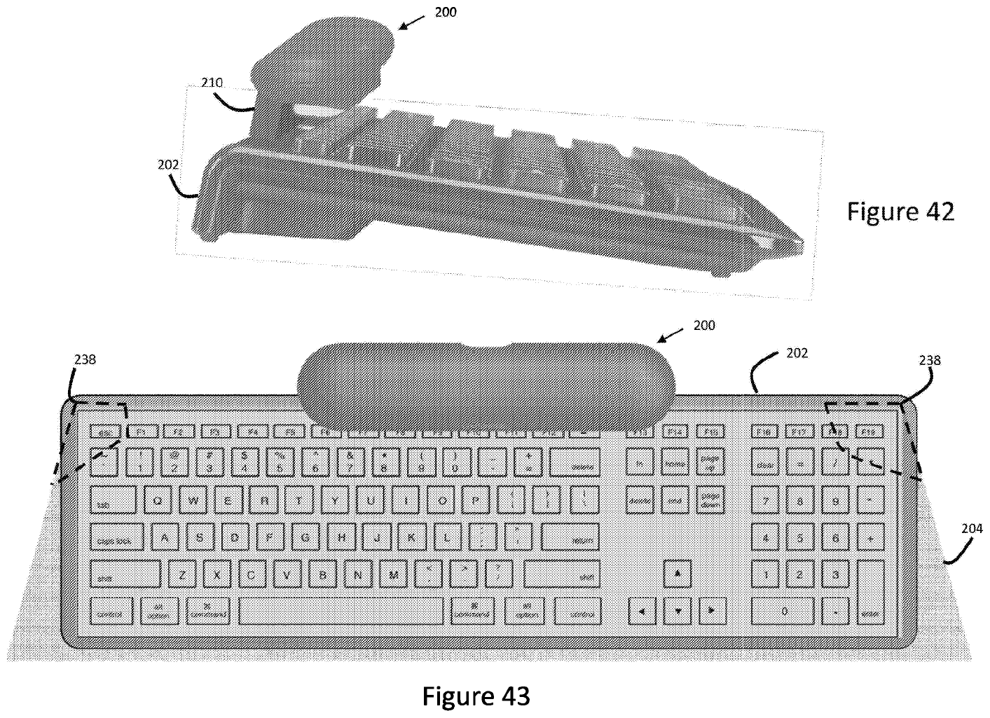

For example, the disinfection device can be configured to provide a UV illumination pattern that produces a relatively uniform intensity pattern at an expected disinfection area where the disinfection device is disposed a few centimeters above the edge of the expected target disinfection area and the opening is angled downward at about 30-45 degrees toward the expected target disinfection area. Of course, the disinfection device can be configured to output a different UV illumination pattern that produces a relatively uniform intensity pattern where the disinfection device is disposed at a different height and different orientation relative to a different expected target disinfection area. That is, for a range of heights and orientations relative to an expected target disinfection area (e.g., a flat surface, an inclined surface, a keyboard and mouse, a keyboard alone, a desk surface with various accessories, a chair, a cabinet, a handle, a cart, a phone, a sink, a countertop, or essentially any other area or surface where the disinfection device may be installed to provide repeatable automated disinfection) the optical lens 226 can impact the UV light such that the UV illumination pattern cast on the expected target disinfection area has uniform intensity. Further, the optical occlusions, if any, e.g., louvers, eyebrow, and occlusion plate, occlusion plate with or without apertures, and occlusion plate with fins reflector or not, and any combination thereof, can occlude a portion of the UV light such that the UV illumination pattern cast on the expected target disinfection area has uniform intensity. It should be understood that uniform intensity does not require all intensity values to be precisely equal, but rather that the intensity at the expected target disinfection area is substantially more uniform than without the optical occlusion or UV lens. In one example, the expected target disinfection area is a keyboard and the UV disinfection device is mounted a few centimeters above the top of the keyboard (for example, as shown in FIGS. 42-43). In this embodiment, the UV disinfection device is configured to output a UV illumination pattern such that minor variances in the contour of the keyboard, for example due to the incline and shape of the keyboard (or due to keyboard kickstands being extended or not) relative to the position and orientation of the UV disinfection device 200 maintain a relatively uniform intensity over the entire expected target UV disinfection area 204 covering the keyboard.

A reflector 222 can be interposed between the circuit board 220 and the UV light source 212 to protect the circuit board 220 from exposure to UV light and reflect UV light toward the opening 208 in the housing. The reflector 222 can include retaining members 221 that pinch the edges of the circuit hoard 220 fixing the reflector in place within the housing 206. The shape, size, reflectivity, and other characteristics of the reflector can vary depending on the application and depending on the characteristics of the other components. In the current embodiments, the reflector 222 forms an arc around the length of the UV source such that a majority of the UV light emitted by the UV source 212 is directed toward aperture 208.

A UV lens 226 having a defined set of optical properties that influence the UV illumination pattern can be disposed on the disinfection apparatus 200 between the UV light source 212 and the target disinfection area 204. In the current disclosure, the UV lens 226 is a flexible UV film 226 that covers the opening 208 and is adhesively coupled or to the internal surface of the lower portion of the housing 216 to seal the internal cavity of the housing 206, held in place by the interaction between the various components within the housing, or otherwise held in place such that it creates a seal between the opening 108 and the internal cavity of the housing 206. The UV lens 226 can be configured to direct UV light from the UV light source 212 through the opening 208 generally, and more specifically through the spacing between the louvers 224.

In some embodiments, the UV lens 226 is a flexible UV film 226 that covers the opening 208 and is adhesively coupled or to the internal surface of the lower portion of the housing 216 to seal the internal cavity of the housing 206, held in place by the interaction between the various components within the housing, or otherwise held in place such that it creates a seal between the opening 108 and the internal cavity of the housing 206. The UV lens 226 can be configured to direct UV light from the UV light source 212 through the opening 208 generally, and more specifically through the spacing between the louvers 224.

The seal provided by the UV lens 226 provides protection. For example, should a component break within the housing 206, the UV film 226 can prevent broken component pieces from falling out as well as gas or liquid from leaking out of the cavity of the housing through the opening 208. The UV film 226 can also prevent unwanted foreign objects or fluids from reaching the components in the internal cavity of the housing 206.

The properties of the UV lens 226 can be selected and enhanced by loading the lens with additives, applying a UV blocking pattern, varying the UV lens material, thickness, shape, layering, surface texture, or any combination thereof. The optical properties for the UV lens can assist in distributing the UV light in a generally uniform UV light pattern across the target disinfection area 204. A more uniform UV light pattern can reduce or prevent UV hot spots, which can cause discoloration or other damage from forming on the UV lens or at the target disinfection area. Some embodiments provide a UV lens with diffusal properties that cause the UV light to diffuse or disperse across the surface of the target disinfection area, such as a user interface surface.

A louver system 224 including a louver frame 228 and directional louvers 227 can be disposed within the opening 208 in the housing 206 to influence the UV illumination pattern output by the disinfection apparatus 200. Referring to the sectional view of FIG. 41, the disinfection device when oriented toward a target disinfection area 204 below and in front of the disinfection device, a user with their eye level at or above the disinfection device does not have a direct line of sight to the UV light source. Further, the dynamic progression of the louvers 227 further limits the direct line of sight such that even where a user's eye level is below the disinfection device, there is no direct line of sight to the UV light source. In addition, the dynamic spacing between the louvers 227 increases the uniformity of the UV illumination pattern by providing more spacing between the louvers toward the front of the disinfection device where the illumination pattern is configured to travel farthest to reach the target disinfection area 104 and less spacing between the louvers 227 toward the rear of the disinfection device where the illumination pattern is configured to travel closest to reach the target disinfection area 204.

The louver system 224 can cover, at least in part, the opening 208. Specifically, the opening 208 can be outlined by a louver frame 228 that can limit the direct line of sight from the UV light source 212 to the user and influence the UV illumination pattern output by the disinfection apparatus 200. The louver frame 228 can include an eyebrow 225 that projects from the front portion of the louver frame 228 in a direction away from the opening 208. Further, the louver frame 228 can support the louvers 227. The louver frame 228 of the illustrated embodiment separates the louvers 227 into three sets with two latitudinal frame sections 230 that span from the rear of the louver frame 228 to the front of the louver frame 228 where the eyebrow 225 is located. The thickness of the latitudinal louver frame sections 230 can vary from the rear to the front such that the thickness frame sections 230 create a flush surface with the bottom of the eyebrow 225. The two latitudinal frame sections 230 split the louvers 224 into three sets, two side sets of four louvers and a middle set of five louvers. The profile of the latitudinal frame sections 230 walls can be curved to influence the UV illumination pattern through the opening 208 and contribute to providing an increase in uniformity of the UV illumination pattern at the target disinfection area. For example, the profile of the latitudinal frame sections 230 in the depicted embodiment are generally concave. The louver frame 228 of the illustrated embodiment also includes longitudinal louver frame sections 232. These longitudinal louver frame sections 232 block UV light from exiting toward the rear of the opening 208, and specifically from the side-rear sections of the opening 108. The two longitudinal frame sections 232 span from one side of the louver frame 228 toward the middle of the louver frame to meet the latitudinal frame sections 230, respectively. The profile of the longitudinal frame sections 230 influence the UV illumination pattern through the opening 208 and contribute to providing an increase in uniformity of the UV illumination pattern at the target disinfection area. For example, the profile of the longitudinal frame sections 230 in the depicted embodiment are generally planar and block UV light closest to the target disinfection area that is apt to receive the highest intensity UV light from the source on account of being closer. Accordingly, the louver frame 228 covers portions of the opening 208 and influences the UV illumination pattern therethrough from the UV light 212. Alternative embodiments can have a different louver frame configuration and different configuration of louvers including additional, fewer, or no louvers and louver frame at all.

A UV opaque nose 235 can occlude a portion of the UV illumination pattern from opening 208. Blocking, reflecting, or absorbing a portion of the UV illumination pattern can increase the uniformity of the intensity of the UV illumination pattern at a target disinfection area. The UV opaque nose 235 is an application specific optic for controlling uniform dose. The nose is removable and replaceable element. Further, it can include a reflective surface for casting design specific patterns.

By positioning the nose 235 near the center of the opening 208, the highest intensity portion of the UV illumination pattern can be occluded. The resultant UV light pattern having two higher intensity side sections with a middle section that has no or low intensity in the middle. This intensity non-uniformity at the output of the disinfection device generally translates to an increase in uniformity at the target disinfection area. While such a UV light pattern may be an improvement over some UV light patterns, the nose 235 can include a variety of different features tailored to increase the overall intensity uniformity of the UV light pattern at the target disinfection area.

The shape of the nose can be tailored to limit UV intensity in the latitudinal direction as well as the longitudinal direction. In the depicted embodiment, it includes a generally isosceles trapezoid UV opaque plastic plate 234 with two UV opaque plastic fins 136 that extend from the trapezoid legs at about a 45 degree angle in a direction away from the midpoint of the disinfection device. Together with the louver frame sections 230, the UV opaque plate 234 occludes much of the middle portion of the UV illumination pattern output from the opening 208.

The plate 234 can include an aperture 242 to limit UV dosage over the occluded area. The aperture 142 can be configured to increase uniformity of intensity of the UV illumination pattern at an expected target disinfection area, for example an expected target disinfection area within a particular distance range away from the disinfection device where the disinfection device is oriented within a particular angle with respect to the target disinfection area. The aperture 242 can be configured by adjusting at least its size, shape, and positioning in the plate 234. For example, the aperture 242 can be latitudinally positioned toward the top third of the trapezoidal plate 234, and be a circular shape having a diameter of about 0.25 millimeters. In alternative embodiments, depending on the application including, for example, the expected position and orientation of the disinfection device, the aperture 242 can have a different size, shape, position, or any combination thereof. In the embodiment illustrated in FIG. 41, an additional aperture 243 is included in an plate 234. The additional aperture 243 is latitudinally positioned toward the bottom third of the plate 234 and has a stadium shape with about double the area of the other aperture 242. This additional aperture can assist in ensuring the target disinfection area receives sufficient UV light to provide disinfection according to any one of a number of UV standards. The angle of an aperture through a surface having a thickness can also affect the UV light, which affects the overall UV illumination pattern from the disinfection device. For example, the occlusion plate 234 while having a generally trapezoid outline, may not be flat, but instead include convex and concave portions formed either by varying the thickness of the structure or the structure including a contour. This can perhaps most easily be seen in the sectional view of FIG. 41, which shows the apertures 243, 242 and the wavy face of the occlusion nose 235. The contouring can also be seen in FIG. 35, depicting the embodiment with one aperture 242. The various characteristics of the aperture(s) can be selected to increase uniform intensity of the UV illumination pattern at an expected target disinfection area for an expected position and orientation of the disinfection device with respect to an expected target disinfection area. In one exemplary embodiment, the aperture 242 characteristics and other disinfection device characteristics provide a 2 μW/cm² intensity level baseline in the middle region of the target disinfection area.

The fins 236 can be formed from a UV reflective material, include a layer of UV reflective material, or have a UV reflective coating on a base substrate that may or may not be UV transmissive such that the inwardly facing side of each fin that faces the opening 208 can reflect UV light incident thereto. The portion of UV light output from the opening 208 incident with the reflective fins influences the UV illumination pattern output from the disinfection device. to shape the UV pattern. For example, as shown in FIG. 43, with the disinfection device disposed and oriented as shown, the UV illumination pattern 204 covers the entire keyboard including the lateral corners 238 near the top of the keyboard 202 that without the reflective fins would not receive UV light. The orientation, shape, and size of the fins can vary depending on the application and the desired UV illumination pattern shape. While the illustrated embodiment includes reflective fins, it should be noted that alternative embodiments of the occlusion nose may not include fins, or may forgo the reflective coating and merely provide occluding fins that do not reflect UV light.

The disinfection apparatus 200 housing 206 can include a coupling mechanism for attaching the disinfection apparatus to an attachment device. For example, the adjustable attachment device can be an adjustable attachment device as described in U.S. Pub. 2015/0297766, filed on Oct. 2, 2013, to Theodore John Cole, entitled PORTABLE LIGHT FASTENING ASSEMBLY, which is hereby incorporated by reference in its entirety. Alternatively, the attachment device can be non-adjustable after installation such that the orientation, height, and positioning of the disinfection apparatus is fixed at installation relative to the target disinfection area. The attachment device 210 may only attach at one end to the disinfection apparatus 210 and may be configured not to attach at the other end to a support structure, but instead form a self-supporting structure. For example, the attachment device can attach at one end to the disinfection device 200 and be configured as a table stand at the other end for placement near a target disinfection area, such as keyboard 202.

The disinfection apparatus 200 can include a circuitry for operation, including power circuitry, control circuitry, sensing circuitry, and communication circuitry. For example, the disinfection apparatus 200 can include circuitry incorporated into the various embodiments of the disinfection device 200 of the present disclosure and variations thereof as described in U.S. Pat. No. 9,242,018 to Cole et al., which is entitled “PORTABLE LIGHT FASTENING ASSEMBLY” and issued on Jan. 26, 2016; U.S. Pat. No. 9,974,873 to Cole et al., which is entitled “UV GERMICIDAL SYSTEM, METHOD, AND DEVICE THEREOF” and issued on May 22, 2018; International application No. PCT/US2019/023842 to Baarman et al., which is entitled “DISINFECTION BEHAVIOR TRACKING AND RANKING” was filed on Jun. 10, 2019; and International application No. PCT/US2019/036298 to Baarman et al., which is entitled “MOBILE DEVICE DISINFECTION” was filed on Jun. 10, 2019, which were previously incorporated by reference in their entireties. As another example, the disinfection apparatus 200 can include circuitry incorporated into the various embodiments of the disinfection device 200 of the present disclosure and variations thereof as described in, U.S. provisional patent application 62/985,976, filed on Mar. 6, 2020, to Baarman et al, entitled “UV DISINFECTION PLATFORM”, which is hereby incorporated by reference in its entirety.

The multilayer UV transmissive film, for example the multilayer UV transmissive medium 804 for the UV disinfection charger, the multilayer UV transmissive medium 1104, or UV film 226 may include two or more layers of materials that have UV transmissive properties. The UV transmissive medium 1104/804/226 can act as a UV transmissive skin or lens that enables transport and distribution of UV light for disinfection. For example, in some embodiments, the different layers can have different UV reflective characteristics, UV absorbing characteristics, UV transmission, or other UV light altering characteristics that cooperate to provide a desired UV light distribution that enables UV light to reach a target disinfection area with a desired intensity range.

The different characteristics of the different layers can be provided in a variety of different ways. The UV multilayer medium can have its optical properties selected by loading the different layers with different (or different amounts of) UV property altering additives, varying the thickness of the layers, applying a UV blocking pattern, varying the layer material type, varying the shape of the different layers, or varying the surface textures of any of the layer surfaces, or any combination thereof. By utilizing multiple layers the UV energy distribution characteristics of the surface can be enhanced such that UV light can reach areas that otherwise would be difficult to reach at the desired intensities, without having to increase the UV source intensity to a range that causes other issues.

While the UV multilayer medium can include any number of layers, many of the embodiments include a set of one or more transport layers and a set of one or more interface layers. A transport layer includes UV light properties that urge UV light input to the layer to transmit along the length of the film. An interface layer includes UV light properties for diffusing the UV light from the transport layer to the interface layer for disinfection. In one embodiment, different layers of the UV transmissive film/medium have different thickness and different loading of additives. In other embodiments, the different layers of the UV transmissive film/medium are made of different material having different UV light properties. The thickness, material, and loading of additives in the interface layer can be such that light received from the transport layer is diffused and reaches the external surface of the interface layer with a sufficient dosage to disinfect the exposed interface surface, including, for example, a device setting on that surface; such as where the exposed interface surface is a UV disinfection charger surface 804 and there is a device setting on that exposed interface surface. The thickness, material, and loading of additives in the transport layer can be such that given a defined UV light input, sufficient light is provided to the interface layer along the entire length of the UV medium/film such that after diffusal by the interface layer, there is sufficient UV light to disinfect the exposed surface (or the surface setting on the exposed surface), even at the distance farthest from the UV light input.

In some embodiments, the multilayer UV transmissive medium 110 may include two or more layers of materials that have different UV properties. Referring to the FIG. 11 embodiment, the outer layer 1106, which has an exposed external surface, is a delivery layer for disinfecting and has particles 1108 that redirect the reflected light from the second, inner layer 1110, which also has the same type of particles 1108, at a lower density. These particles may be quartz or other translucent materials for UVC but with facets that refract the light outward. This second layer 1108 is a distribution layer and is designed with enough reflective materials to have some energy available at the end of the distance of material while providing sufficient UV energy to the outer layer 1106 to disinfect the exposed surface of the outer layer 1106. Put another way, one layer of the multilayer UV transmissive film 1102 has a higher density of reflective particles while the other has a lower level of reflective particles and is used primarily for distribution of the energy to the first layer and has a lower density of reflective particles.

The FIG. 11 sectional view can also similarly describe a UV multilayer transmissive medium for use in connection with a UV disinfection charger, such as depicted in FIG. 29. Instead of being applied to a touch screen or keypad, the multilayer UV transmissive medium can be applied to a substrate or other surface of the UV disinfection charger. The substrate 1100 may have reflective properties to encourage the UV light to distribute along the UV-C multilayer transmissive medium. UV light can be input into the UV transmissive multilayer medium in a variety of different ways. In some embodiments, UV light is shined from UV source 808 toward the surface 804 such that the UV light disinfects the exposed surfaces of a device or devices disposed on the surface. In addition, UV source 808 incident to the UV transmissive multilayer medium 804 is distributed across the surface to the underside of the one or more devices disposed on the surface 804, disinfecting those surfaces. In alternative embodiments, the UV source 808 can light pipe or otherwise distribute UV light to the edge of the UV transmissive medium 804 where the UV source housing 828 meets the housing 826 surface. The UV multilayer transmissive medium 804 may be surrounded by a UV transmissive gasket 830. The gasket can encourage UV light to travel along the edge of the UV transmissive surface and enables greater distribution of UV light over the UV multilayer transmissive medium 804. The gasket can be sandwiched between the UV disinfection charger housing 826 and the UV multilayer transmissive medium 804. The gasket can be made from a UV transmissive material and may include any of the enhancements described herein of the different UV layers of the multilayer UV transmissive medium. For example, the gasket itself may include light altering properties from being loaded with light altering additives. Further the thickness, texture, and material of the gasket can be selected to provide the desired UV light transmission properties.

In another embodiment, depicted in FIG. 12, a UV transmissive film 1202 is applied to the surface of a tube 1200, such as a cable or medical device. The tube can have an internal cavity 1204. UV input light 1206 may be directed toward the edge of the UV transmissive film 1202 from a UV source (not shown) either installed in or on the tube 1200 or external to the tube 1200. The UV input light 1206 may be incident with an edge of the UV transmissive film 1200, with a portion of an edge of the UV transmissive film, or the UV input light 1206 may be incident with the UV transmissive tubing 1200. The UV transmissive film 1202 can be co-molded or extruded over tubing that has the reflective properties mixed within the material. It can also be printed for specific inverse square law filtering and energy dispersion. FIG. 12 shows tubing with a co-extruded casing that enables some of the energy within the primary tube 1200 to be broadcast through and out of the casing. The primary casing may also have a printed pattern to further the distribution pattern limiting exposure and more evenly distributing the UVC energy. As UV light travels along the UV transmissive tube 1200, some of the light becomes incident with the UV transmissive film 1202 and strikes the microparticles 1208, which provide a UV light diffusion effect that disperses the output UV light 1210 along the external surface of the UV transmissive film to a greater degree than otherwise. The length of the arrows representing the output light arrows 1210 are relatively short, representing that the UV light exiting the UV transmissive film has been diffused and the intensity of the UV light quickly decreases as it travels away from the tubing due to the diffusal effects of the UV transmissive film 1202 with diffusal enhancing additive particles.

F. UV Housing

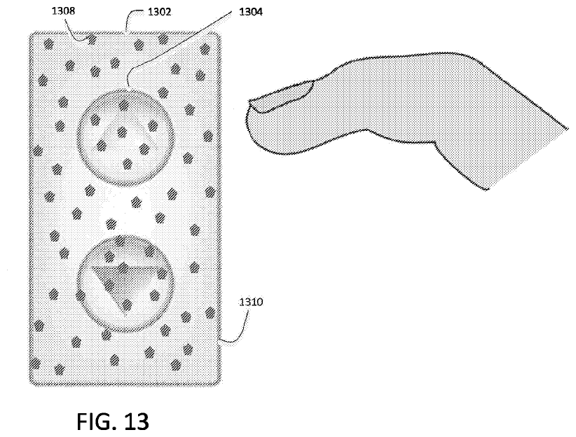

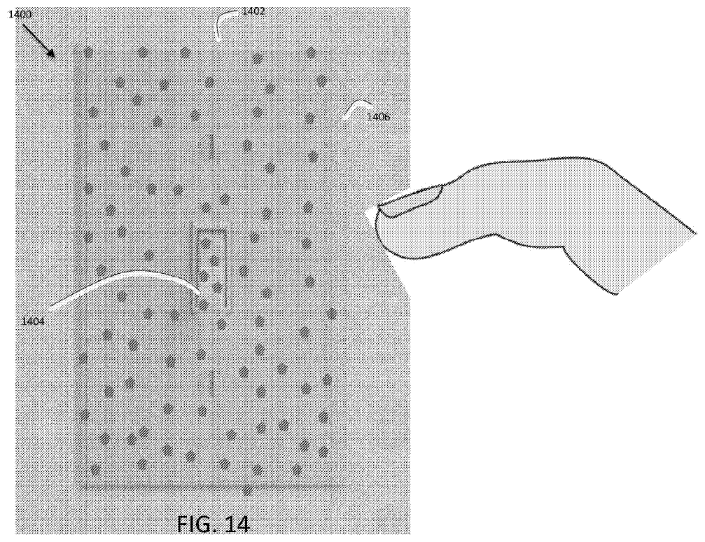

FIGS. 13-16 show examples of UV transmissive housings of various user interface devices. A UV transmissive housing can improve UV disinfection properties of a user interface device by enabling UV light to travel along and be guided by the UV transmissive housing to reach areas that otherwise might not be completely disinfected by a UV disinfection device transmitting UVC light to the device. The optical properties of the UV transmissive housing can be selected by loading the housing with additives, applying a UV blocking pattern, varying the housing material type, thickness, shape, layering, or surface texture, or any combination thereof.

FIG. 13 shows an elevator plate 1302 with up and down buttons 1304. The elevator plate and buttons can be loaded with additives to enhance the optical characteristics of the housing. In addition, the thicknesses of the elevator plate 1302 can be varied in order to accommodate and guide UV light about the plate. For example, the corners and edges 1310 of the elevator plate can have different thicknesses. Generally, the thinner the material, the more UV transmissive. Accordingly, by varying the thickness of the plate, the UV transmission characteristics of the plate can be changed and the UV light pattern that results from UV light being shined on or toward the plate can be changed. The combination of the variable thickness of the elevator plate and the loading of additives, for example that diffuse UV light, work together to improve the UV disinfection properties of the elevator plate. That is, in response to a particular pattern of UVC light shined on the elevator plate, the UVC light is distributed throughout the material to the exposed surfaces and provide effective and improved disinfection of the elevator plate. The specific characteristics of the elevator plate can be arrived at by UV light modeling software, for example the density and type of additives loaded into the elevator plate material and the different thicknesses of the elevator plate can be selected based on desired UV intensity measurements.

FIG. 14 shows a light switch 1400 including a light switch cover plate 1402 and a light switch 1404. The light switch cover plate 1402 and switch 1404 can be loaded with additives to enhance the optical characteristics of the light switch. In addition, the thicknesses of the light switch plate 1402 can be varied in order to accommodate and guide UV light about the plate. For example, the corners and edges 1406 of the plate can have different thicknesses. Generally, the thinner the material, the more UV transmissive. Accordingly, by varying the thickness of the plate, the UV transmission characteristics of the light switch 1400 can be changed and the UV light pattern that results from UV light being shined on or toward the plate can be changed. The combination of the variable thickness of the light switch plate and the loading of additives, for example that diffuse UV light, work together to improve the UV disinfection properties of the light switch 1400. That is, in response to a particular pattern of UVC light shined on the light switch 1400, the UVC light is distributed throughout the material to the exposed surfaces of the light switch and provide effective and improved disinfection of the light switch 1400. The specific characteristics of the light switch 1400 can be arrived at by UV light modeling software, for example the density and type of additives loaded into the light switch and the different thicknesses of the plate can be selected based on desired UV intensity measurements. In addition, or instead, a printed filter can be provided on the light switch. For example, the light switch plate can include a UV diffusion coating with additives that diffuse UV light when struck.

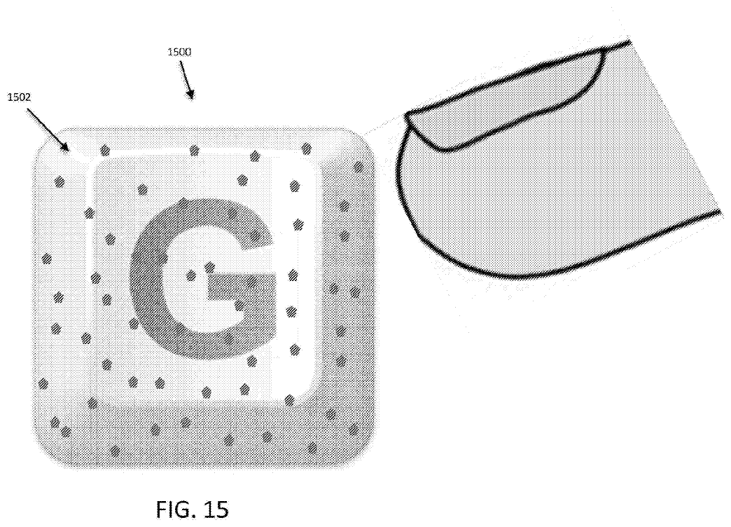

FIG. 15 shows a keyboard key 1500. The housing of the key 1500 can be loaded with additives to enhance the optical characteristics of the light switch or a UV diffusion or transmissive coating can be applied to the external surface of the key. In addition, the thicknesses of the key 1500 can be varied in order to accommodate and guide UV light about the exposed surface of the key. For example, the corners and edges 1502 of the key can have different thicknesses. Generally, the thinner the material, the more UV transmissive. Accordingly, by varying the thickness of the key, the UV transmission characteristics of the keyboard key 1500 (and entire keyboard) can be changed and the UV light pattern that results from UV light being shined on or toward the key can be changed. The combination of the variable thickness of the key and the loading of additives, for example that diffuse UV light, work together to improve the UV disinfection properties of the key 1500. That is, in response to a particular pattern of UVC light shined on the key 1500, the UVC light is distributed throughout the material to the exposed surfaces of the key and provide effective and improved disinfection. The specific characteristics of the key 1500 can be selected by the use of UV light modeling software, for example the density and type of additives loaded into the key and the different thicknesses can be selected based on desired UV intensity measurements on the surface.

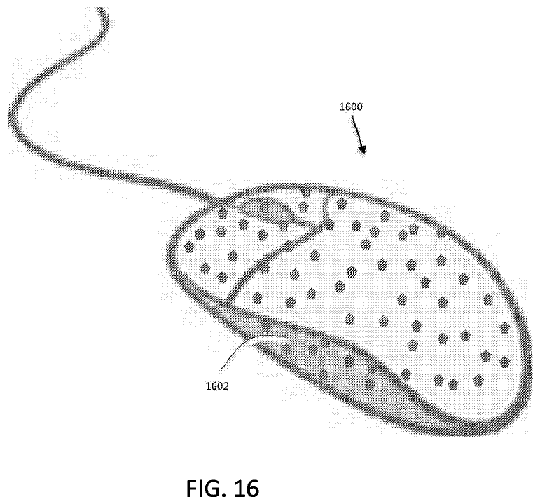

FIG. 16 shows a mouse 1600. The housing of the mouse 1600 can be loaded with additives to enhance its optical characteristics or a UV transmissive coating can be applied to the external surface of the mouse. In addition, the thicknesses of the mouse 1500 can be varied in order to accommodate and guide UV light about the exposed surface of the mouse. For example, the corners and edges 1602 of the mouse can have different thicknesses. Generally, the thinner the material, the more UV transmissive. Accordingly, by varying the thickness of the mouse, the UV transmission characteristics of the mouse can be changed and the UV light pattern that results from UV light being shined on or toward the mouse can be changed. The combination of the variable thickness of the mouse and the loading of additives, for example that diffuse UV light, work together to improve the UV disinfection properties of the mouse 1600. That is, in response to a particular pattern of UVC light shined on the mouse 1600, the UVC light is distributed throughout the material to the exposed surfaces and provides effective and improved disinfection. The specific characteristics of the mouse 1600 can be selected by the use of UV light modeling software, for example the density and type of additives loaded into the key and the different thicknesses can be selected based on desired UV intensity measurements on the surface.

Directional terms, such as “vertical,” “horizontal,” “top,” “bottom,” “upper,” “lower,” “inner,” “inwardly,” “outer” and “outwardly,” are used to assist in describing the invention based on the orientation of the embodiments shown in the illustrations. The use of directional terms should not be interpreted to limit the invention to any specific orientation(s).

The above description is that of current embodiments of the invention. Various alterations and changes can be made without departing from the spirit and broader aspects of the invention as defined in the appended claims, which are to be interpreted in accordance with the principles of patent law including the doctrine of equivalents. This disclosure is presented for illustrative purposes and should not be interpreted as an exhaustive description of all embodiments of the invention or to limit the scope of the claims to the specific elements illustrated or described in connection with these embodiments. For example, and without limitation, any individual element(s) of the described invention may be replaced by alternative elements that provide substantially similar functionality or otherwise provide adequate operation. This includes, for example, presently known alternative elements, such as those that might be currently known to one skilled in the art, and alternative elements that may be developed in the future, such as those that one skilled in the art might, upon development, recognize as an alternative. Further, the disclosed embodiments include a plurality of features that are described in concert and that might cooperatively provide a collection of benefits. The present invention is not limited to only those embodiments that include all of these features or that provide all of the stated benefits, except to the extent otherwise expressly set forth in the issued claims. Any reference to claim elements in the singular, for example, using the articles “a,” “an,” “the” or “said,” is not to be construed as limiting the element to the singular.

1. A UV disinfection device comprising:
a housing;
a UV-C source installed within said housing, said UV-C source configured to emit UV-C light for disinfecting a target disinfection area outside of the UV disinfection device;
a reflector installed within said housing, said UV-C reflector configured to reflect UV-C light emitted from said UV-C source;
a lens joined with the housing and positioned between said UV-C source and the target disinfection area outside of the UV disinfection device, wherein said lens is configured to generate a generally uniform UV-C light pattern.

2. The UV disinfection device of claim 1 wherein the UV-C lens is a fluoropolymer material loaded with SiO₂ microparticles.

3. The UV disinfection device of claim 1 wherein the UV-C lens has a variable thickness.

4. The UV disinfection device of claim 1 wherein said UV-C lens is a fluoropolymer material loaded with SiO₂ microparticles and said UV-C lens has a variable thickness, which in combination contribute to said UV-C lens configuration that said relatively uniform UV-C light pattern.

5. The UV disinfection device of claim 1 wherein an internal surface of the UV-C lens includes a UV blocking pattern.

6. The UV disinfection device of claim 1 wherein an external surface of the UV-C lens is textured.

7.-25. (canceled)

26. A UV disinfection device comprising:
a housing;
a UV-C source installed within said housing, said UV-C source configured to emit UV-C light for disinfecting a target disinfection area outside of the UV disinfection device;
a reflector installed within said housing, said UV-C reflector configured to reflect UV-C light emitted from said UV-C source;
a lens joined with the housing and positioned between said UV-C source and the target disinfection area outside of the UV disinfection device, wherein said lens is configured to generate a generally uniform UV-C light pattern;
a composite lens joined with the housing and positioned between said UV-C source and the target disinfection area outside of the UV disinfection device, wherein said composite lens is configured to produce a UV light pattern with reduced intensity closer to the UV source and provide an overall energy output pattern to a target distance and dosage with a generally uniform intensity pattern, the composite lens including:
a primary lens with a first UVC transmissivity level;
a secondary lens with a second UVC transmissivity level greater than the first UVC transmissivity level;
wherein the primary and secondary lens cooperate to reduce or prevent UV hot spots during normal us of the UV disinfection device.

27. The UV disinfection device of claim 26 wherein the UV source is positioned offset from the target disinfection area and the disinfection device, including the composite lens, is positioned at a non-perpendicular angle relative to the target disinfection area.

28. The UV disinfection device of claim 26 wherein the properties of the primary and secondary lens material are selected by one or more of loading the primary and secondary lenses with additives, applying different UV blocking patterns to the primary and secondary lenses, varying one or more of material type, thickness, shape, and surface texture of the primary and secondary lenses.

29. The UV disinfection device of claim 26 wherein the lens material of the primary and secondary lenses are loaded with different amounts of additives providing different light diffusal properties.

30. The UV disinfection device of claim 26 wherein the primary and secondary lenses are joined and wherein the position of the secondary lens is offset with respect to the primary lens in order to generate a generally uniform UV light pattern when the composite lens is installed in a UV disinfection device oriented at an angle with respect to a target disinfection surface.

31. The UV disinfection device of claim 26 wherein the secondary lens is an insert held in place by the primary lens with a pair of fingers that at least partially surround the secondary lens.

32. The UV disinfection device of claim 26 wherein the primary lens is generally cuboid shaped and the secondary lens is generally in the shape of an elliptical cylinder.

33. The UV disinfection device of claim 26 wherein the primary lens includes a supplemental prism that juts inward away from the target disinfection area configured to creating a more uniform light distribution at the target disinfection area.

34.-94. (canceled)
